(19) United States
(12) Patent Application Publication
(10) Pub. No.: US 2026/0116838 A1
(43) Pub. Date: Apr. 30, 2026

(54) NEOCARZILIN DERIVATIVES AS ANTIMETASTATIC AGENTS

(71) Applicant: Purdue Research Foundation, West Lafayette, IN (US)

(72) Inventors: Elizabeth Ivy Parkinson, West Lafayette, IN (US); Jason Hanna, West Lafayette, IN (US); Noah Moriarty, Lafayette, IN (US); Annaleigh Powell, Chapel Hill, NC (US)

(73) Assignee: Purdue Research Foundation, West Lafayette, IN (US)

(21) Appl. No.: 19/307,338

(22) Filed: Aug. 22, 2025

Related U.S. Application Data
(60) Provisional application No. 63/711,951, filed on Oct. 25, 2024.

(51) Int. Cl. C07C 49/227; A61K 31/121

(52) U.S. Cl. CPC C07C 49/227; A61K 31/121

(57) ABSTRACT

Compounds that inhibit vesicle amine transport 1 (VAT1); compositions comprising the same; and their use for inhibiting metastasis in cancer.

### CROSS-REFERENCE TO RELATED APPLICATIONS

This application claims priority to U.S. provisional patent application No. 63/711,951, which was filed Oct. 25, 2024, and which is hereby incorporated by reference in its entirety.

### STATEMENT OF GOVERNMENT SUPPORT

This invention was made with government support under CA023168 and GM125620 awarded by the National Institutes of Health. The government has certain rights in the invention.

### TECHNICAL FIELD

The present disclosure relates to compounds that are inhibitors of vesicle amine transport 1 (VAT1). In particular, the disclosure relates to Neocarzilin A (NCA) derivatives, compositions comprising the same, and their use for inhibiting metastasis.

### BACKGROUND

This section introduces aspects that may help facilitate a better understanding of the disclosure. Accordingly, these statements are to be read in this light and are not to be construed as admissions about what is or is not prior art.

Metastasis is the most common cause of death by cancer. However, few to no antimetastasis agents exist. Migrastatic compounds target fundamental cell motility pathways to inhibit migration and metastasis. This contrasts with the more traditional anticancer cytostatic compounds, which target cancer cell proliferation/survival. One advantage of migrastatics not targeting proliferative pathways is that they generally have much lower toxicity and, thus, fewer adverse side effects compared to traditional chemotherapeutics (Molecules 2022, 27 (7); Trends Cancer 2023, 9 (4), 293-308). In addition, resistance is less common for migrastatics since they do not have a selective pressure to produce resistant subpopulations.

Vesicle amine transport protein-1 (VAT1) is an understudied protein recently implicated in cancer cell migration in several cancers, including glioblastoma, hepatocellular carcinoma, gastric cancer, and breast cancer (BC) (Neoplasma 2021, 68 (2), 416-422; Oncol Rep 2019, 42 (4), 1422-1430; ACS Cent Sci 2019, 5 (7), 1170-1178). Based on the work reported in Benton, A. et al., Molecular Cancer Research 2024, 22 (9), 879-890, there is an overexpression of VAT1 in angiosarcoma (AS) cases. In AS, breast cancer, and glioblastoma, depletion of VAT1 has been shown to impair migration in cell culture (Oncol Rep 2019, 42, 1422-1430; ACS Cent Sci 2019, 5, 1170-1178). These data suggest that inhibition of VAT1 is a promising anti-metastatic target. However, until recently, no chemical inhibitors have been able to probe the druggability of VAT1.

Natural products play a vital role in the development of lifesaving drug molecules. Their structural novelty, diversity, and complexity make them unparalleled for novel therapeutic development. However, due to their structural complexity and natural scarcity, it is often difficult to conduct a comprehensive medicinal chemistry study around the active natural product to improve its potency, selectivity, and other drug properties. Natural products are also valuable sources of covalent protein modifiers due to the presence of electrophilic groups. The application of natural products and their derivatives for the development of VAT1 inhibitors is an approach that has previously been shown to be fruitful.

It is an object of the present disclosure to provide a compound that is an inhibitor of vesicle amine transport protein-1(VAT1) for inhibiting cancer metastasis with improved pharmacokinetic properties and no toxicity. This and other objects and advantages, as well as inventive features, will be apparent from the detailed description.

### SUMMARY

Provided is a compound selected from:

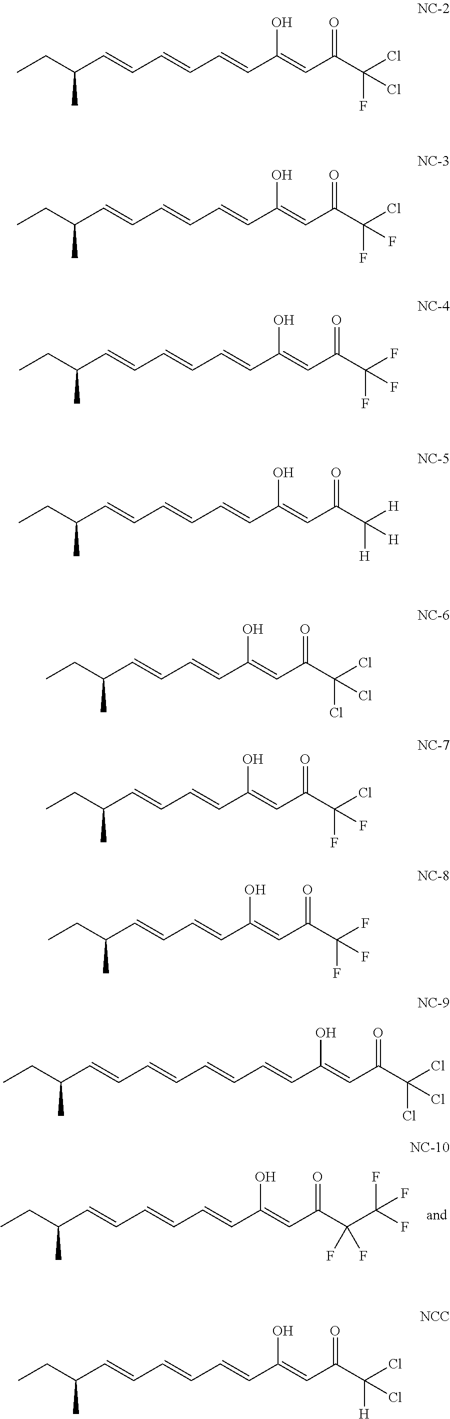

or a pharmaceutically acceptable salt or hydrate thereof.

Provided is a pharmaceutical composition comprising a compound selected from:

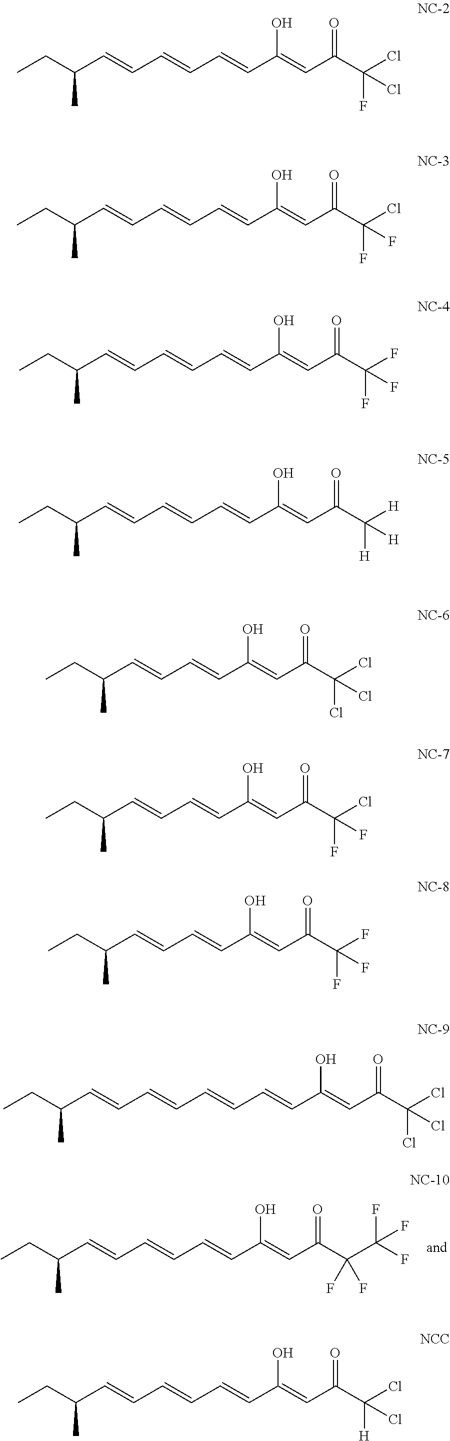

or a pharmaceutically acceptable salt or hydrate thereof, and a pharmaceutically acceptable carrier, excipient, or diluent.

In some embodiments, the pharmaceutical composition further comprises another compound, which is a therapeutic agent. The therapeutic agent can be an anti-cancer agent.

Further provided is a method of inhibiting vesicle amine transport protein-1 (VAT1) in a patient in need thereof, which method comprises administering to the patient an inhibitory effective amount of the above-described pharmaceutical composition, whereupon the VAT1 is inhibited in the patient. In some embodiments of the method, the pharmaceutical composition can be administered orally or parenterally. In some embodiments of the method, the patient has cancer. The cancer can be breast cancer, glioblastoma, hepatocellular carcinoma, or angiosarcoma. In some embodiments of the method, the metastasis of the cancer is inhibited by inhibiting VAT1.

Provided is a compound of the formula:

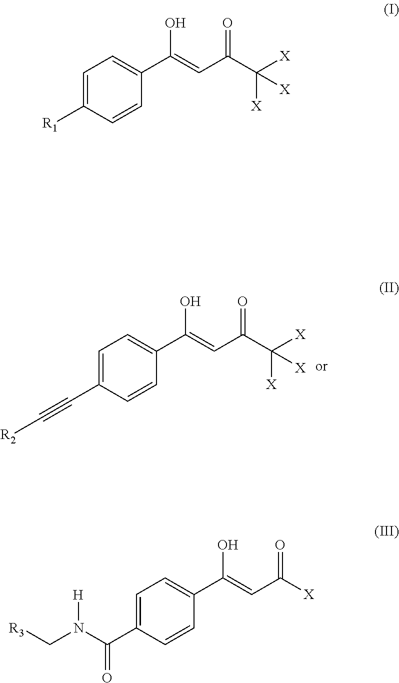

or a pharmaceutically acceptable salt or hydrate thereof,
wherein:
each R₁, R₂, and R₃ is independently C₁-C₆ alkyl, aryl, heteroaryl, or heterocyclyl, wherein each R₁, R₂, and R₃ can be optionally independently substituted with at least one of C₁-C₆ alkyl, aryl, heteroaryl, and heterocyclyl; and
X is hydrogen or halogen.

In some embodiments of the compound of formula (I), the R₁ is:

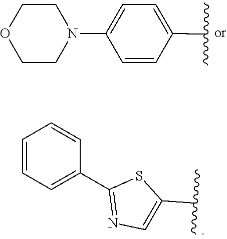 [SMILES: *c1ccc(N2CCOCC2)cc1 ; *c1cnc(-c2ccccc2)s1]

In some embodiments of the compound of formula (II), the R₂ is:

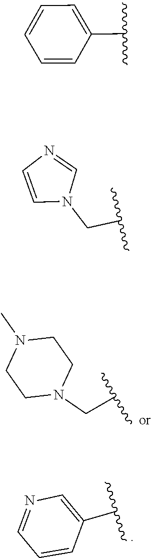

In some embodiments of the compound of formula (II), the R₃ is:

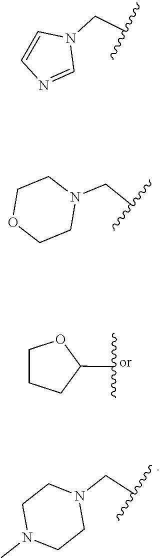

In some embodiments, X is halogen.

### BRIEF DESCRIPTION OF THE DRAWINGS

The above and other objects, features, and advantages of the present disclosure will be apparent when the description is read in conjunction with the drawings.

FIG. 1 shows structures of FDA-approved drugs with reversible covalent warheads and the natural product Neocarzilin A (NCA).

FIG. 2A shows previously studied trifluoromethyl ketones and their characteristic hydration. The HDAC inhibitor 8b and the clinically evaluated human leukocyte elastase inhibitor ZD-0892 have previously been shown to equilibrate to the hydrate species in water at different ratios depending on the substituents surrounding the warhead.

FIG. 2B shows the equilibrium of ketone to hydrate in aqueous media for NCA and the compounds NC-7 to NC-9. n=3; error is the standard deviation. Significance was determined using a student's t-test with each additional fluorine, resulting in a significantly higher amount of hydrate (P<0.0001).

FIG. 3A shows the transwell migration activity of NCA and other compounds. Representative images from three independent transwell migration assays of vehicle (DMSO), NCA, and compounds NC-3, NC-4, and NC-7 treated MDA-MB-231 cells. All treatments were performed at 1 μM.

FIG. 3B shows the quantification of the migration activity of NCA and compounds NC-3, NC-4 and NC-7. Experiments were performed at least three independent times. Dots indicate results from independent experiments. Bars indicate average results, and the error shown is the standard deviation. Student's t-test was performed to compare each treated sample to the vehicle (DMSO) control. **P<0.01; ***P<0.001.

FIG. 4 shows a pharmacokinetics analysis of NCA and other compounds. The compound was administered at 25 mg/kg to mice (n=3) via oral gavage. Plasma concentration was monitored over a period of 4 hours. The dotted lines indicate 10 μM (top) and 1 μM (bottom). The table shows that due to the compound still being in the distribution phase, NC-3 and NC-4 exhibited the shortest half-life. However, the compounds exhibited a clear improvement in the absorption and systemic concentrations of the compound compared to NCA, highlighting their potential for studying VAT1 inhibition in vivo. kel=elimination rate constant, t½=half-life, M_{RT}=mean retention time, tₘₐₓ=time at which maximum plasma concentration was reached, Cₘₐₓ=maximum plasma concentration.

FIG. 5 shows the representative images from anti-migratory scratch and wound healing assays for compounds NCA, NC-2-NC-10. Images were taken at t=0 and 24 hand the area of the scratch was quantified using ImageJ software. Images are representative of 16 different scratch planes and for three independent experiments. Quantifications of % migration were normalized to the DMSO control.

FIG. 6 shows kinetic solubility measurements of NCA, NC-2, and NC-3 using laser nephelometry with the solubility determination as the intersection between the linear soluble and insoluble ranges. Representative of three or more independent experiments.

FIG. 7 shows kinetic solubility measurements of NC-4, NC-5, and NC-6 using laser nephelometry with the solubility determination as the intersection between the linear soluble and insoluble ranges. Representative of three or more independent experiments.

FIG. 8 shows kinetic solubility measurements of NC-7 and NC-8 using laser nephelometry. Solubility was determined to be greater than 500 due to its high solubility and, therefore, high variability in the insoluble linear range. This is also due to the challenge in making a concentrated enough stock to go beyond 2500 μM. It also shows the kinetic solubility measurements of NC-9 using laser nephelometry. Due to the poor solubility of NC-9 and the inconsistency in the soluble linear ranges, the solubility was determined to be less than 10 μM. Representative of three or more independent experiments.

FIG. 9 shows kinetic solubility measurements of NC-10 using laser nephelometry with the solubility determination as the intersection between the linear soluble and insoluble ranges. Representative of three or more independent experiments.

FIG. 10 shows the IC₅₀ curve for the compound NCA as analyzed from GraphPad Prism (ver. 10.3.0). IC₅₀ reported as the mean from all reps plus or minus the standard deviation.

FIG. 11 shows the IC₅₀ curve for the compound NC-2 as analyzed from GraphPad Prism (ver. 10.3.0). IC₅₀ reported as the mean from all reps plus or minus the standard deviation.

FIG. 12 shows the IC₅₀ curve for the compound NC-3 as analyzed from GraphPad Prism (ver. 10.3.0). IC₅₀ reported as the mean from all reps plus or minus the standard deviation.

FIG. 13 shows the IC₅₀ curve for the compound NC-4 as analyzed from GraphPad Prism (ver. 10.3.0). IC₅₀ reported as the mean from all reps plus or minus the standard deviation.

FIG. 14 shows the IC₅₀ curve for the compound NC-5 as analyzed from GraphPad Prism (ver. 10.3.0). IC₅₀ reported as the mean from all reps plus or minus the standard deviation.

FIG. 15 shows the IC₅₀ curve for the compound NC-6 as analyzed from GraphPad Prism (ver. 10.3.0). IC₅₀ reported as the mean from all reps plus or minus the standard deviation.

FIG. 16 shows the IC₅₀ curve for the compound NC-7 as analyzed from GraphPad Prism (ver. 10.3.0). IC₅₀ reported as the mean from all reps plus or minus the standard deviation. Due to its low toxicity NC-7 has an incomplete IC₅₀ curve and is noted as greater than 100 μM.

FIG. 17 shows the IC₅₀ curve for the compound NC-8 as analyzed from GraphPad Prism (ver. 10.3.0). IC₅₀ reported as the mean from all reps plus or minus the standard deviation.

FIG. 18 shows the IC₅₀ curve for the compound NC-10 as analyzed from GraphPad Prism (ver. 10.3.0). IC₅₀ reported as the mean from all reps plus or minus the standard deviation.

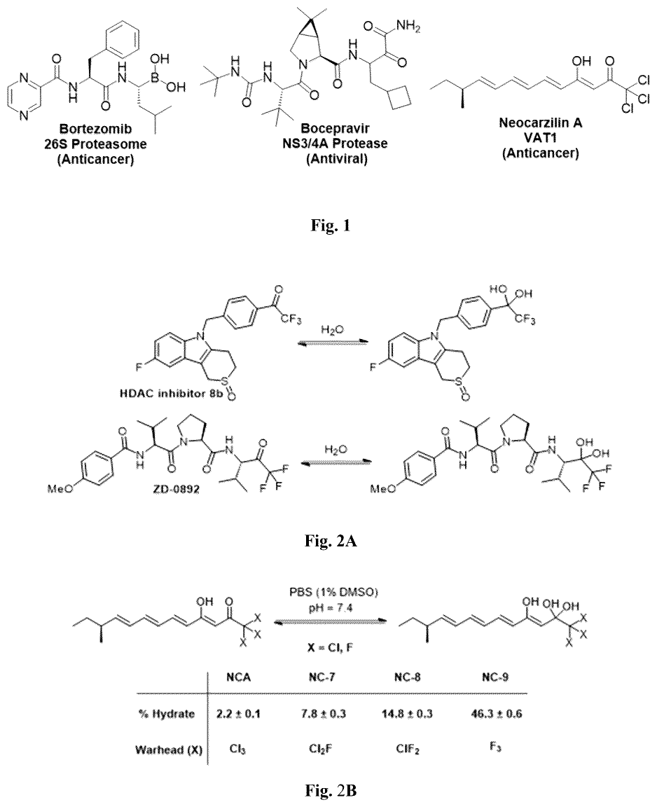

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “VAT 1” refers to vesicle amine transport 1 (VAT 1, also known as vesicle amine transport protein 1 homolog, synaptic vesicle membrane protein VAT-1 homolog, FLJ20230, membrane protein of cholinergic synaptic vesicles, and EC 1).

The term “covalent inhibitors” refers to inhibitors that form a covalent bond, which can be a strong covalent bond, with the target protein.

The present disclosure is predicated, at least in part, on the role of natural products that can covalently modify cellular target proteins in developing lifesaving drug molecules. Neocarzillin A (NCA) is a natural product discovered from Streptomyces carzinostaticus, which inhibits metastasis in cell culture (Tetrahedron Lett, 1992, 33, 7547-7550). Chemical proteomic studies identified vesicle amine transport protein-1 (VAT1), a protein that contributes to cancer cell migration, as one of the covalent targets of NCA among ˜10 total protein targets. NCA inhibits the metastasis. However, NCA likely exhibits antiproliferative activity through polypharmacology, as the knockdown of VAT1 shows little effect on proliferation. Its antimigratory activity is due to the inhibition of VAT1. However, NCA has poor solubility and pharmacokinetic properties, which makes in vivo evaluation challenging. Previous attempts at formulation proved difficult, requiring both dimethyl sulfoxide (DMSO) and Solutol HS 15 as solubilizing agents (ACS Cent Sci 2019, 5, 1170-1178). Also, it does not selectively inhibit VAT1, which results in off-target toxicities. Many angiosarcoma (AS) patients (20-40%) present with metastatic disease at diagnosis, and half with localized disease will develop metastases. Unfortunately, there is no clear standard of care treatment plan for metastatic AS patients.

In view of the above, the present disclosure provides compounds inspired by NCA, which are VAT1 inhibitors with improved solubility and selectivity for antimigratory activity. The compounds can inhibit metastasis with low-to-no toxicity and improved pharmacokinetic properties. The compounds comprise halomethyl ketone (A), wherein X is halogen selected from chlorine or fluorine, as their warhead.

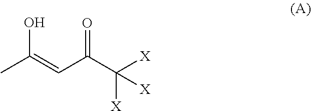

For covalent or reversible covalent inhibitors, the reactivity of the warhead is a critical component. The compounds comprise electrophilic Michael acceptors, such as an a, 0-unsaturated carbonyl group, which can form a covalent bond with a reactive functional group in the target protein, such as VAT1. The compounds can covalently inhibit VAT 1, which is a critical mediator of AS metastasis.

Provided is a compound selected from:

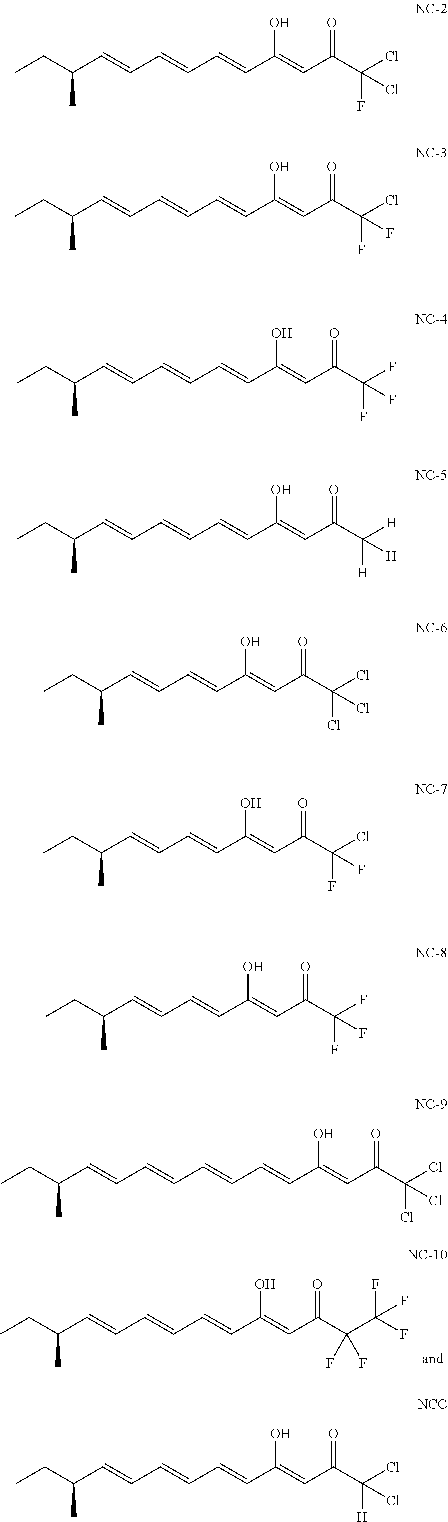

or a pharmaceutically acceptable salt or hydrate thereof.

Provided is a pharmaceutical composition comprising a compound selected from:

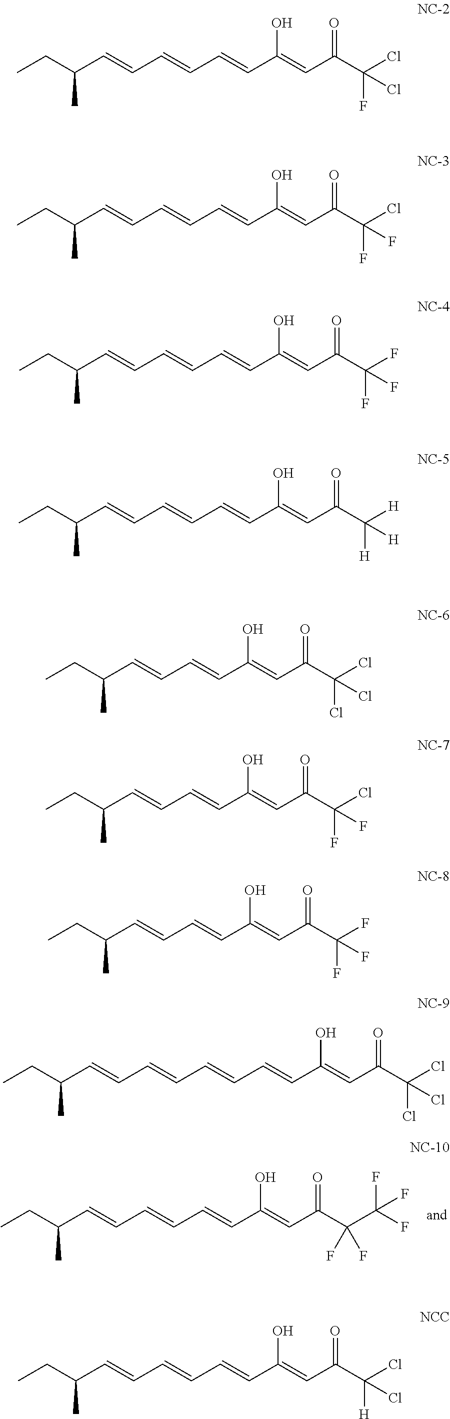

or a pharmaceutically acceptable salt or hydrate thereof, and a pharmaceutically acceptable carrier, excipient, or diluent.

In some embodiments, the compounds NC-2, NC-3, NC-4, NC-5, NC-6, NC-7, NC-8, NC-9, NC-10 and NCC comprise the formula (A)

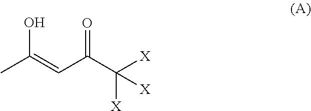

as their pharmacophore and the trihalomethyl ketone, such as chlorodifluoromethyl ketone group as the electrophilic warhead. The warhead can bind VAT1 through a reversible covalent interaction group. This warhead can improve the solubility and antimigratory activity of the compounds. The improved solubility can result in a better pharmacokinetic profile over the known compound NCA. The chlorodifluoromethyl ketone warhead can target threonine/serine. It can be more selective for hard nucleophiles such as threonine/serine compared to softer cysteine nucleophiles. Changing the length of the alkene region can affect the solubility. A longer alkene region can result in less aqueous solubility. The shorter alkene chain compound NC-6 has approximately 4-fold greater aqueous solubility than NCA. In some embodiments, the compounds can have greater than 25-fold improvements in solubility. In some embodiments, the compounds can have about 25-fold improvements in solubility. In some embodiments, the compounds can have greater than 30-fold improvements in the antimigratory to antiproliferative bioactivity ratio. In some embodiments, the compounds can have about a 30-fold improvement in the antimigratory to antiproliferative bioactivity ratio.

In some embodiments, the pharmaceutical composition can further comprise another compound, which is a therapeutic agent. The therapeutic agent can be any known anti-cancer agent. The anti-cancer agent includes a natural product or a natural product-derived covalent drug.

Examples of the known natural products or natural product-derived covalent drugs that can be used to treat cancer include, but are not limited to, aspirin, penicillin, finasteride, orlistat, fumaderm, triptolide, minnelide, sonolisib (PX-866), E6201, dimethylamino parthenolide, bardoxolone methyl, ludongnin, eriocalyxin B, jungermannenone C, enmein, adenanthin, and oridonin.

Provided is a pharmaceutical composition comprising (i) a compound selected from NC-2, NC-3, NC-4, NC-5, NC-6, NC-7, NC-8, NC-9, NC-10 and NCC or a pharmaceutically acceptable salt or hydrate of any of the foregoing, (ii) a one or more additional therapeutic agent, and (iii) at least one pharmaceutically acceptable carrier, excipient, or diluent. The carrier, excipient, or diluent can vary based on the particular route of administration (see, e.g., Remington's The Science and Practice of Pharmacy, 23rd ed. (2020)).

Provided is a method of inhibiting vesicle Amine Transport-1 (VAT1) in a patient in need thereof, which method comprises administering the patient an inhibitory effective amount of a pharmaceutical composition comprising a compound selected from NC-2, NC-3, NC-4, NC-5, NC-6, NC-7, NC-8, NC-9, NC-10 and NCC or a pharmaceutically acceptable salt or hydrate of any of the foregoing, and a pharmaceutically acceptable carrier, excipient, or diluent, whereupon the VAT1 is inhibited in the patient. In some embodiments of the method, the patient has cancer.

In some embodiments of the method, the cancer is breast cancer, glioblastoma, hepatocellular carcinoma, or angiosarcoma. The pharmaceutical composition can be administered orally or parenterally.

Further provided is a method of inhibiting metastasis in a patient by inhibiting VAT 1, which method comprises administering the patient an inhibitory effective amount of a pharmaceutical composition comprising a compound selected form NC-2, NC-3, NC-4, NC-5, NC-6, NC-7, NC-8, NC-9, NC-10 and NCC or a pharmaceutically acceptable salt or hydrate of any of the foregoing and a pharmaceutically acceptable carrier, excipient, or diluent, whereupon the metastasis is inhibited in the patient. In some embodiments of the method, the patient has cancer. In some embodiments of the method, the cancer is breast cancer, glioblastoma, hepatocellular carcinoma, or angiosarcoma. The compound or pharmaceutical composition can be administered orally or parenterally.

Further provided is a method of treating or inhibiting cancer associated with metastatic diseases in a patient comprising administering the patient an effective amount of a pharmaceutical composition comprising a compound selected from NC-2, NC-3, NC-4, NC-5, NC-6, NC-7, NC-8, NC-9, NC-10 and NCC or a pharmaceutically acceptable salt or hydrate of any of the foregoing and a pharmaceutically acceptable carrier, excipient, or diluent, whereupon the cancer is treated or inhibited in the patient. In some embodiments of the method, the cancer is breast cancer, glioblastoma, hepatocellular carcinoma, or angiosarcoma.

Further provided is a compound of the formula:

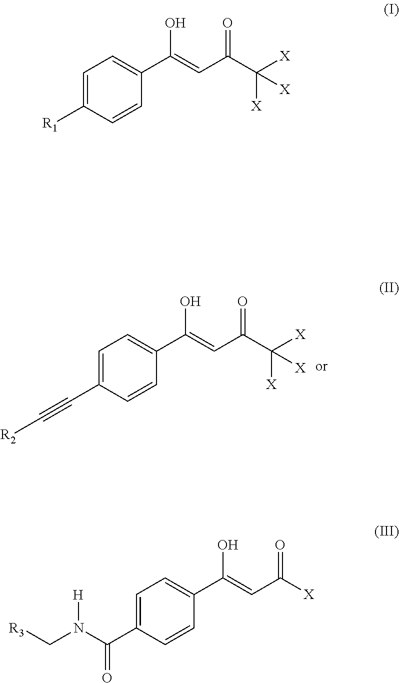

or a pharmaceutically acceptable salt or hydrate thereof,
wherein:
each R₁, R₂, and R₃ is independently C₁-C₆ alkyl, aryl, heteroaryl, or heterocyclyl, wherein each R₁, R₂, and R₃ can be optionally independently substituted with at least one of C₁-C₆ alkyl, aryl, heteroaryl and heterocyclyl; and
X is hydrogen or halogen.

In some embodiments of the compound of formula (I), the R₁ is:

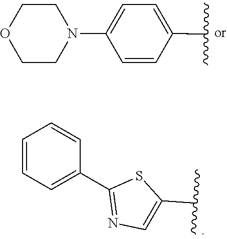 [SMILES: *c1ccc(N2CCOCC2)cc1 ; *c1cnc(-c2ccccc2)s1]

In some embodiments of the compound of formula (II), the R₂ is:

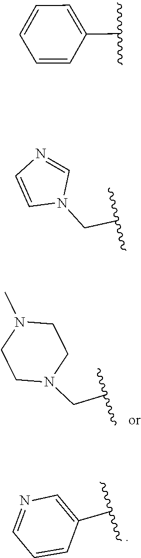

In some embodiments of the compound of formula (III), the R₃ is:

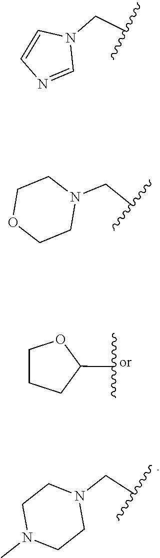

In some embodiments, X is halogen.

The compounds can inhibit metastasis by inhibiting VAT1. Provided is a pharmaceutical composition comprising a compound of formula (I), (II), or (III) or a pharmaceutically acceptable salt or hydrate of any of the foregoing and a pharmaceutically acceptable carrier, excipient, or diluent.

Further provided is a method of inhibiting vesicle amine transport-1 (VAT1) in a patient in need thereof comprising administering to the patient an inhibitory effective amount of (i) a compound of formula (I), (II) or (III), or (ii) a pharmaceutically acceptable salt or hydrate of (i), or (iii) a pharmaceutical composition comprising (i) or (ii), whereupon the VAT1 is inhibited in the patient.

In some embodiments of the method, the patient has cancer. The cancer can be breast cancer, glioblastoma, hepatocellular carcinoma, or angiosarcoma. The compound can be administered orally or parenterally.

Still further provided is a method of inhibiting metastasis in a patient by inhibiting VAT1 comprising administering the patient an inhibitory effective amount of (i) a compound of formula (I), (II) or (III), or (ii) a pharmaceutically acceptable salt or hydrate of (i), or (iii) a pharmaceutical composition comprising (i) or (ii), whereupon the metastasis is inhibited in the patient. In some embodiments, the patient has cancer. In some embodiments of the method, the cancer can be breast cancer, glioblastoma, hepatocellular carcinoma, or angiosarcoma. The compound can be administered orally or parenterally.

### Synthesis of Compounds

The NCA and its derivatives, such as compounds NCC, and NC-2-NC-10 were synthesized using processes (Schemes 1A-1C) similar to as reported in ACS Cent Sci 2019, 5 (7), 1170-1178 and Tetrahedron Lett 1992, 33 (49), 7551-7552, which are incorporated herein by reference for their teaching regarding the same.

Methyl 4-bromocrotonate and triethyl phosphite were reacted neat in an Arbuzov reaction to afford phosphonate 1 in quantitative yield on 10 gram scale. S-2-methyl butanol was oxidized to the aldehyde 2 using Dess-Martin Periodinane (DMP). It was observed that the yields were more consistent with DMP than manganese dioxide (MnO₂), which was used in the reported process, likely due to inconsistent activation of MnO₂ and the volatility of aldehyde 2. 1 was subjected to lithium bistrimethylsilyl amide (LiHMDS) to afford the phosphonate ylide intermediate, which was subjected to Horner Wadsworth Emmons (HWE) olefination with aldehyde 2 to afford the diene methyl ester 3 in 50-70% yield over two steps on gram scale. Quantitative reduction of diene methyl ester 3 with diisobutylaluminum hydride (DIBAlH) afforded alcohol 4. Alcohol 4 was directly oxidized to aldehyde 5 using DMP to react with the ylide triphenylphosphoranylidene 2-propanone in a Wittig olefination to afford the common intermediate ketone 6 in 50-60% yield over two steps on a 500 mg scale. Direct reduction to the aldehyde with DIBAlH was also successfully attempted, as has been previously demonstrated, but yields were more consistent when using multiple equivalents to reduce to the alcohol followed by oxidation to the aldehyde with DMP. Ketone 6 was reacted with LiHMDS to afford the enolate, which was acylated directly with (CX₃CO)₂O or CX₃COCl (X═H, Cl, F) to afford NCA, NCC, NC-3, NC-4 and NC-5 in 40-75% yield on a 100 mg scale. For NC-2, dichlorofluoroacetic acid was reacted with oxalyl chloride in the presence of catalytic DMF to afford the acid chloride 7, which was directly subjected to the enolate of ketone 6 to afford NC-2.

The truncated alkene compounds (NC-6 to NC-9) were accessed in a similar fashion. Briefly, ethyl bromoacetate was reacted with triethyl phosphite neat in an Arbuzov reaction to afford phosphonate 8 in quantitative yield on a I0 gram scale. S-2-methylbutanol was oxidized to the aldehyde with DMP and reacted with the phosphonate ylide derived from 8, reacting with LiHMDS. This afforded the α,β-unsaturated ester 9 in 50-70% yields over two steps on a gram scale. The ester was reduced with DIBAlH to afford alcohol 10 in quantitative yield. Alcohol 10 was oxidized to the aldehyde and reacted with the ylide triphenylphosphoranylidene 2-propanone to afford the common intermediate ketone 11 in 40-60% yield over two steps on a 650 mg scale.

Ketone 11 was reacted with LiHMDS to afford the enolate that was directly acylated with (CX₃CO)₂O (X═Cl, F) to afford the truncated compounds NC-6 to NC-8 in 15-55% yield on a 35-100 mg scale.

Synthesis of the elongated compound NC-9 started from diene ester 3, which was reduced to intermediate diene alcohol 4. Alcohol 4 was oxidized to the aldehyde using DMP and reacted with the phosphonate ylide, resulting from reacting 8 with LiHMDS in an HWE olefination reaction. This afforded the triene ester 12 in 48% yield over two steps on a 400 mg scale. Ester 12 was reduced to alcohol 13 using DIBAlH in quantitative yield. 13 was oxidized to the aldehyde and subjected to a Wittig reaction with the commercially available ylide triphenylphosphoranylidene 2-propanone to afford the elongated ketone 14 in 37% yield over two steps on a 230 mg scale. Ketone 14 was reacted with LiHMDS to afford the enolate and subsequently acylated with trichloroacetic anhydride to afford NC-9 in 77% yield on a 100 mg scale.

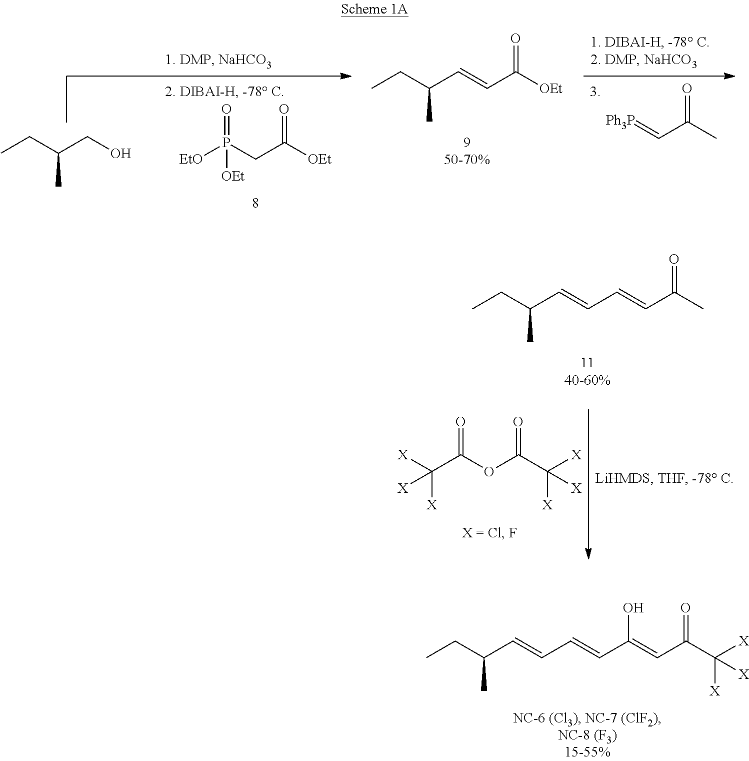

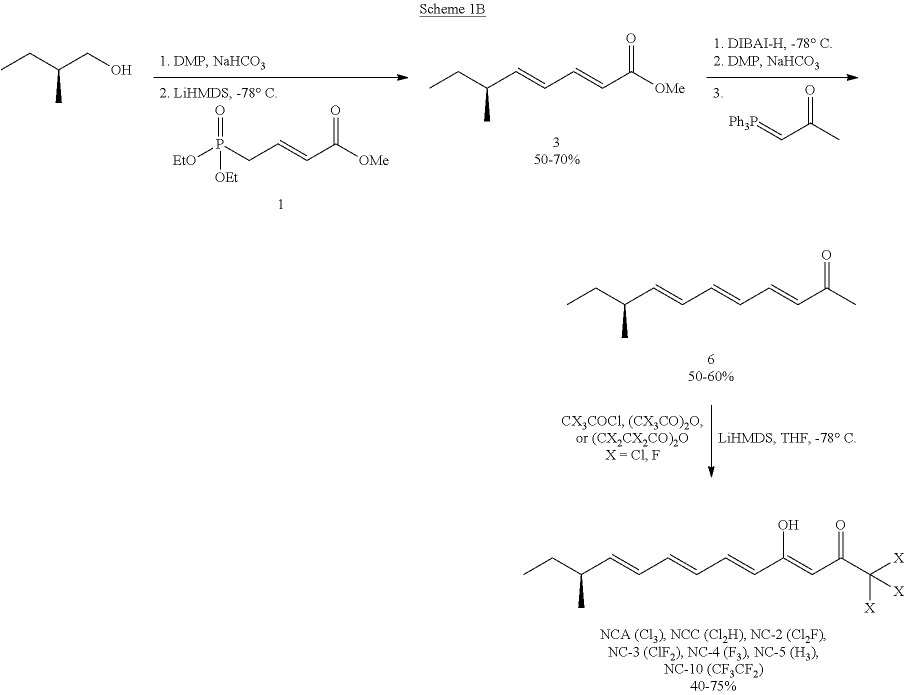

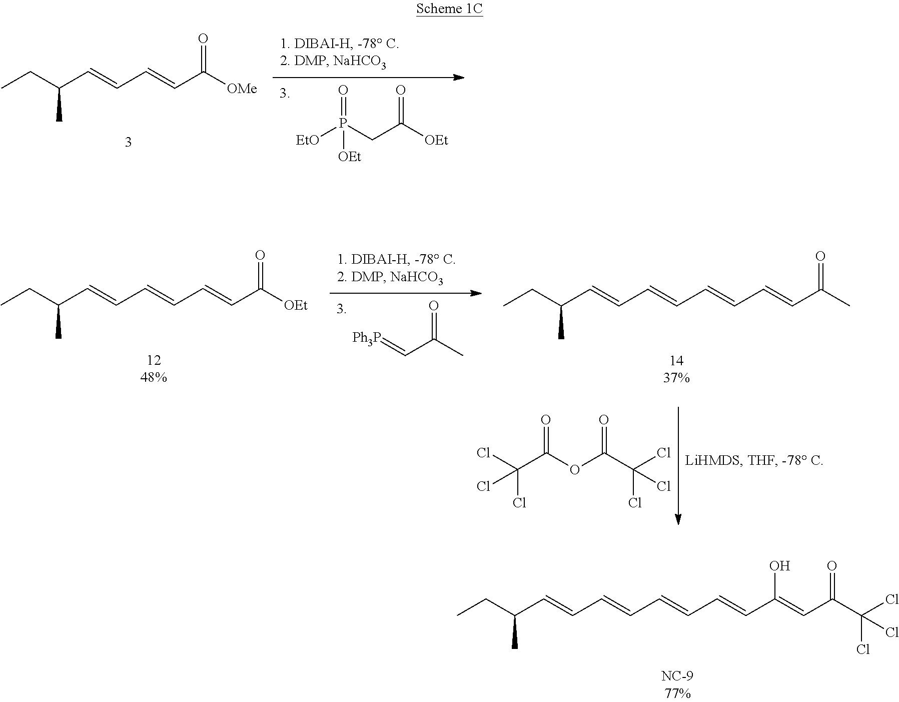

The compounds of formulae (I), (II), and (III) can be prepared using the synthesis described in Scheme 2.

### Synthesis of Compounds of Formula (I), (II) and (III)

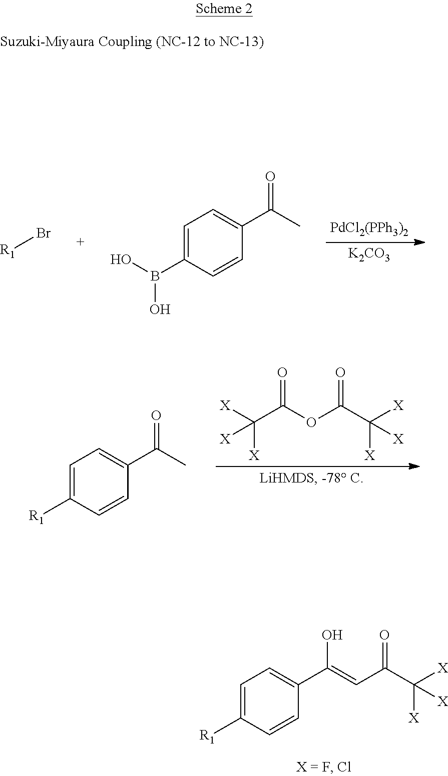

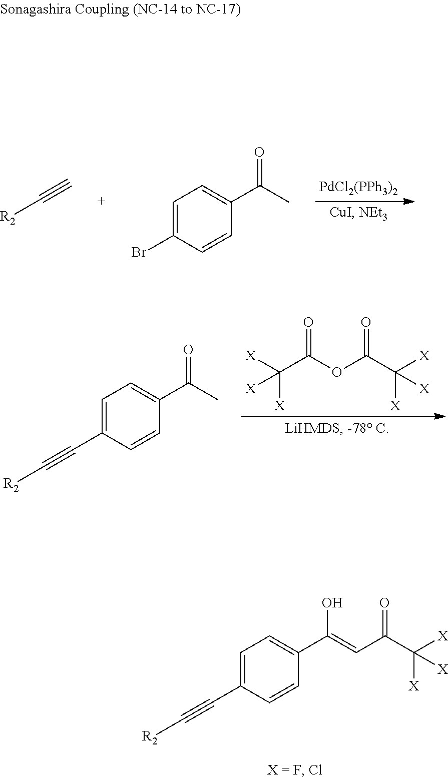

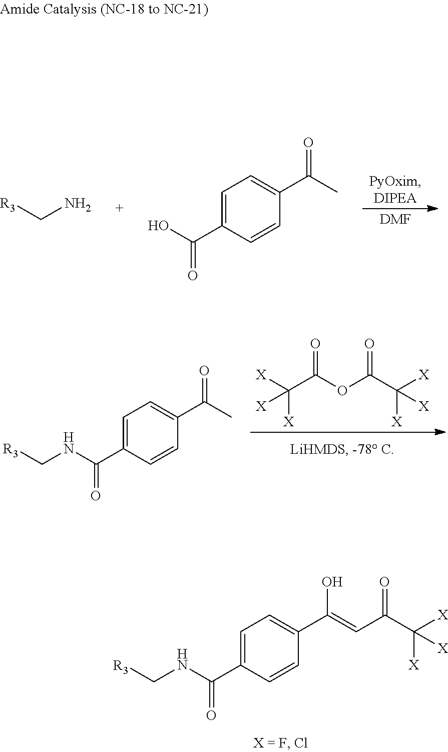

### Analysis of Aqueous Solubility

It was reported that aqueous solubility significantly correlates with bioavailability and pharmacokinetics/pharmacodynamics. Models for predicting bioavailability and PK parameters developed by Kasugi et al. Mol Pharm 2021, 18 (3), 1071-1079 and Parrott et al. Mol Pharm 2022, 19 (11), 3858-3868 found that aqueous solubility was an important parameter for predicting oral bioavailability. Additionally, in the development of novobiocin derivatives, it was found that optimization of solubility was one of the most important determinants to improve the pharmacokinetics of their lead molecule (Bioorg Med Chem 2023, 92, 117381). Improvements in solubility over solithromycin and gepotidacin also led to improved bioavailability and clearance pharmacokinetic parameters. It was hypothesized that more soluble compounds NC-3 and NC-4 would exhibit a more favorable pharmacokinetics profile than NCA and thus would make them better tools for studying VAT1 inhibition in vivo.

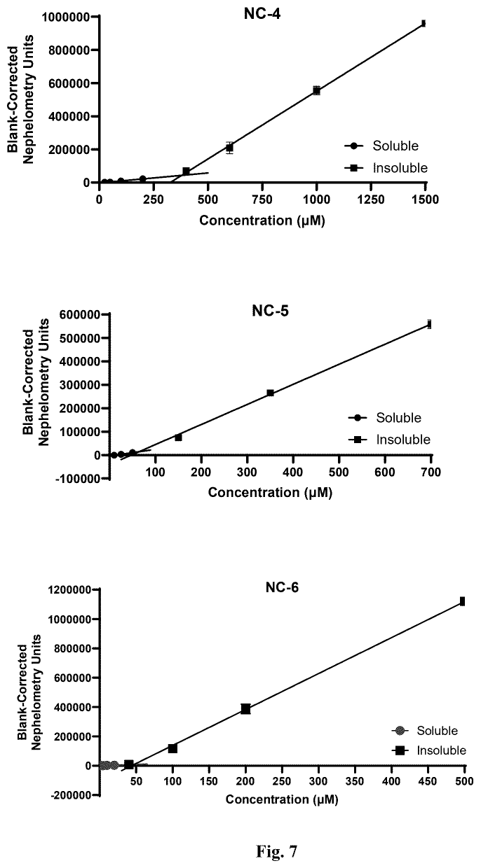

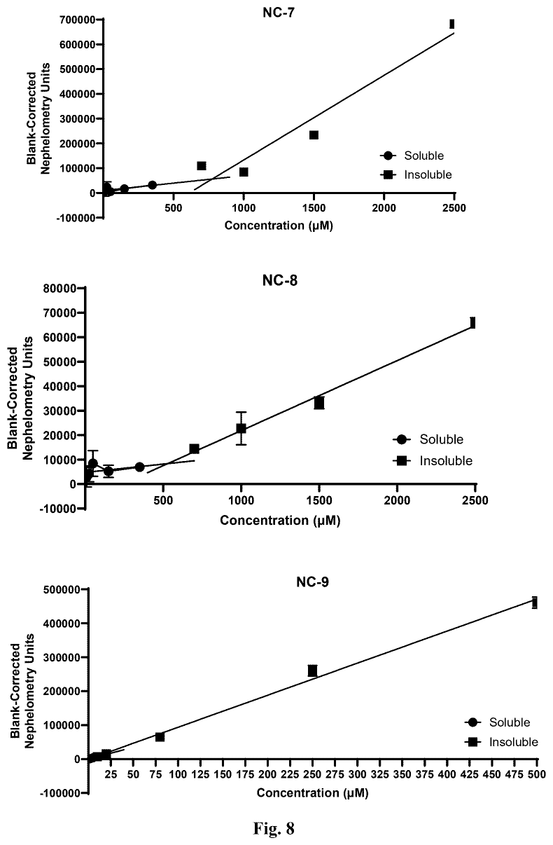

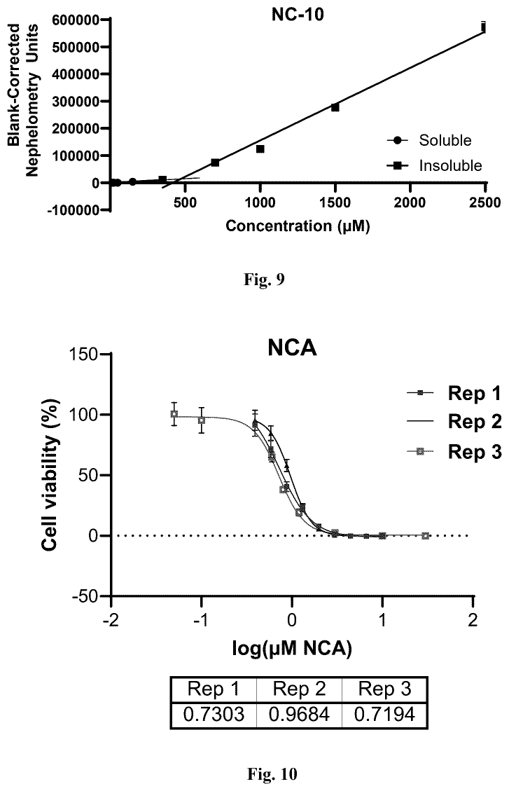

The aqueous solubility of the compounds was identified using laser nephelometry (Table 1 and FIGS. 6-9). NCA was very poorly soluble (12 μM). The shorter alkene NC-6 has approximately 4-fold greater aqueous solubility than NCA, while the longer alkene region results in less aqueous solubility (<10 μM). Due to the very poor solubility of the longer alkene compound, further elongated compounds were not explored. The fluorinated warheads dramatically improved solubility, leading to up to 30-fold greater solubility for the trifluoro derivative NC-4 compared to the parent NCA, with a stepwise increase in the solubility being observed for each additional fluorine on the warhead. During the solubility studies, the increases in observed solubility were far greater than those predicted by SwissAMDE. FIGS. 2A-2B shows that trifluoromethyl ketones exist in equilibrium with their hydrated forms. These hydrated forms are predicted to be more water soluble compared to their ketone counterparts (Table 2).

To better understand the predominant form of the compounds in aqueous buffer, it was set out to quantify the amount of hydrate that exists in equilibrium with the ketone using liquid chromatography tandem mass spectrometry (LCMS/MS). NCA, NC-2, NC-3, or NC-4 was pre-equilibrated in phosphate buffered saline (PBS), and the ratio of ketone to hydrate was quantified (FIG. 2B). The trichloromethyl ketone existed primarily as the ketone, with little hydrate (˜2%) being observed. Chlorines are not as electron-withdrawing as fluorines, leaving the electrophilic carbonyl carbon less polarized than derivatives with fluorines. As such alpha-chlorinated ketones have not previously been determined to be in equilibrium with their respective hydrates. Interestingly, all of the fluorinated warheads were capable of forming a substantial amount of hydrate, with each additional fluorine resulting in statistically significantly higher amounts of hydrate. For NC-4, there were nearly equal amounts of ketone (55%) and hydrate (45%) in the equilibrium. When taking into account the amount of hydrate, the predicted aqueous solubility for the fluorinated warheads is much more accurately predictable using SwissADME (Table 2).

TABLE 1
Summary of the predictions and results for the activities and solubilities of NCA and derivatives.

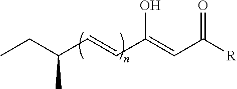

| | | | Docking | | | % | |
|---|---|---|---|---|---|---|---|
| | Linker | Warhead | Score* | Solubility | | Migration | Selectivity |
| Compound | Length (n) | (R) | (kcal/mol) | (μM) | IC₅₀ (μM) | at 1 μM | Index** |
| NCA | 3 | COCCl₃ | −45.36 | 12 ± 2 | 0.8 ± 0.1 | 57 ± 25 | 1 |
| NCC | 3 | COCHCl₂ | −43.52 | 45 ± 6 | 10 ± 4 | 72 ± 14 | 10 |
| NC-2 | 3 | COCCl₂F | −46.94 | 30 ± 5 | 16 ± 7 | 90 ± 18 | 13 |
| NC-3 | 3 | COCClF₂ | −48.31 | 158 ± 11 | 43 ± 27 | 83 ± 8 | 37 |
| NC-4 | 3 | COCF₃ | −45.55 | 360 ± 22 | 27 ± 4 | 80 ± 5 | 24 |
| NC-5 | 3 | COCH₃ | −51.67 | 63 ± 5 | >100 | 95 ± 17 | 75 |
| NC-6 | 2 | COCCl₃ | −42.47 | 43 ± 4 | 4.1 ± 2.6 | 72 ± 14 | 4 |
| NC-7 | 2 | COCClF₂ | −44.84 | 43 ± 4 | >100 | 89.5 ± 0.4 | 80 |
| NC-8 | 2 | COCF₃ | −39.74 | >500 | 75 ± 15 | 92 ± 9 | 58 |
| NC-9 | 4 | COCCl₃ | −47.00 | <10 | ND | 51 ± 6 | 1 |
| NC-10 | 3 | COCF₂CF₃ | −44.78 | 453 ± 22 | 28 ± 7 | 86 ± 20 | 23 |

*Covalent MM-GBSA docking scores; **Selectivity Index = ratio of % migration at 1 μM and the IC₅₀ for each derivative in comparison to NCA; IC₅₀ and migration studies were performed with MDA-MB-231 cells; Migration studies are scratch assays; ND = not determined.

Table 2 shows predicted solubilities from SwissADME calculations for NCA, NC-2, NC-3, and NC-4 as well as their hydrate confomers. Based on the determined ratios of hydration for each derivative, a weighted solubility prediction can be done by accounting for the level of hydration for each compound. Weighted solubility predictions provided a better estimate of compound solubility than solubility predictions of ketone alone.

TABLE 2

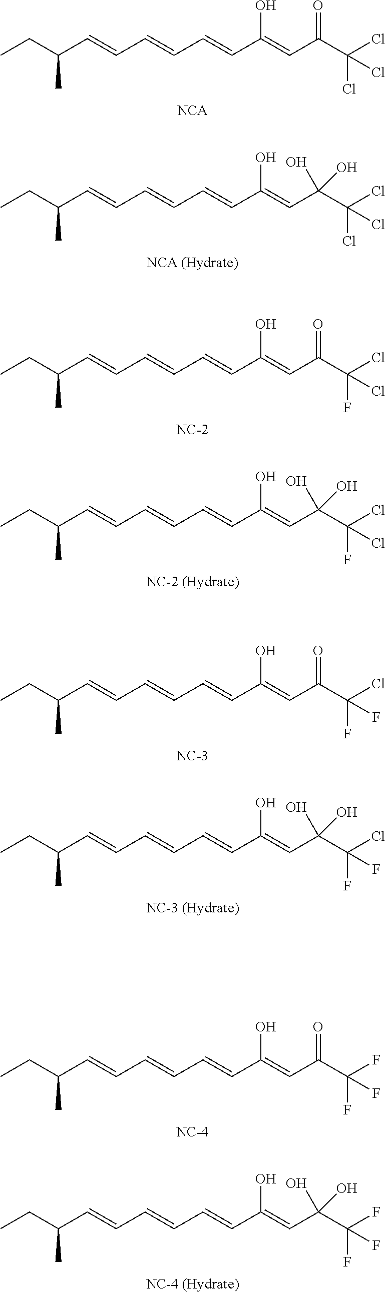

| Solubility | | NCA | | NC-2 | | NC-3 | | NC-4 |
|---|---|---|---|---|---|---|---|---|
| Prediction | NCA | (hydrate) | NC-2 | (hydrate) | NC-3 | (hydrate) | NC-4 | (hydrate) |
| Predicted | 8.51 | 71.0 | 15.5 | 129 | 28.1 | 231 | 50.4 | 420 |
| (μM) | | | | | | | | |
| % Confomer | 97.8 | 2.2 | 92.2 | 7.8 | 85.2 | 14.8 | 53.7 | 46.3 |
| Weighted | 9.88 | — | 24.35 | — | 58.13 | — | 221.52 | — |
| Solubility | | | | | | | | |
| (μM) | | | | | | | | |
| Experimental | 12 | — | 30 ± | — | 158 ± | — | 360 ± | — |
| Solubility | | | 5 | | 11 | | 22 | |
| (μM) | | | | | | | | |

Table 3 shows the docking scores and solubility predictions. Rx is R₁, R₂ and R₃ in the structures shown in Scheme 2.

| | | | Docking | | |
|---|---|---|---|---|---|
| | | | Score | Predicated | |
| Compound | | | (MMGBSA | Solubility | |
| No. | Formula | Rₓ | Kcal/mol) | (UM) | X |
| NCA | — | — | −48.46 | 8.5 | Cl |
| NC-12 | 1 | 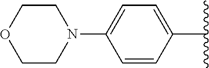 [SMILES: *c1ccc(N2CCOCC2)cc1] | −51.93 | 9.6 | F |
| NC-13 | 1 | 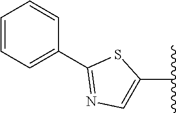 [SMILES: *c1cnc(-c2ccccc2)s1] | −54.95 | 1.7 | F |
| NC-14 | 2 | 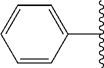 [SMILES: *c1ccccc1] | −47.89 | 5.8 | F |
| NC-15 | 2 | 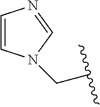 [SMILES: *Cn1ccnc1] | −58.15 | 114 | F |
| NC16 | 2 | 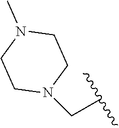 [SMILES: *CN1CCN(C)CC1] | −53.54 | 114 | F |
| NC-17 | 2 | 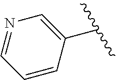 [SMILES: *c1cccnc1] | −51.68 | 26.8 | F |
| NC-18 | 3 | 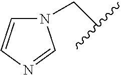 [SMILES: *Cn1ccnc1] | −60.86 | 676 | F |
| NC-19 | 3 | 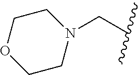 [SMILES: *CN1CCOCC1] | −54.92 | 791 | F |
| NC-20 | 3 | 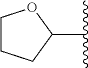 [SMILES: *C1CCCO1] | −50.91 | 340 | F |
| NC-21 | 3 | 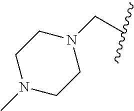 [SMILES: *CN1CCN(C)CC1] | −49.85 | 513 | F |

The term “substituted” refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halo (e.g., F, Cl, Br, and I); an oxygen atom, such as in groups like hydroxyl groups, alkoxy groups, aryloxy groups, aralkyloxy groups, oxo(carbonyl) groups, and carboxyl groups (including carboxylic acids, carboxylates, and carboxylate esters); a sulfur atom, such as in groups like thiol groups, alkyl and aryl sulfide groups, sulfoxide groups, sulfone groups, sulfonyl groups, and sulfonamide groups; a nitrogen atom, such as in groups like amines, azides, hydroxylamines, cyano, nitro groups, N-oxides, hydrazides, and enamines; and other heteroatoms, e.g., in various other groups.

Non-limiting examples of substituents, which can be bonded to a substituted carbon atom (or other atom, such as nitrogen) include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, C₁-C₆ alkyl, acyl, C₃-C₈ cycloalkyl, C₆-C₁₂ aryl, C₁-C₆ aralkyl, heterocyclyl, heteroaryl, or C₁-C₆ heteroarylalkyl, wherein alkyl, acyl, cycloalkyl, aryl, aralkyl, heterocyclyl, heteroaryl, or heteroarylalkyl or, two R groups attached to a nitrogen atom or two adjacent nitrogen atoms can, together with the nitrogen atom or atoms to which they are attached, form a heterocyclyl ring, which can be mono- or independently multi-substituted.

The terms “optionally substituted” and “optional substituents” indicate that the groups in question are either unsubstituted or substituted with one or more of the above-mentioned substituents.

When the groups in question are substituted with more than one substituent, the substituents may be the same or different. When used with the terms “independently”, “independently are,” and “independently selected from,” the groups in question may be the same or different. Certain of the herein defined terms may occur more than once in the structure and, upon such occurrence, each term shall be defined independently of the other.

The term “alkyl” refers to substituted or unsubstituted straight-chain and branched alkyl groups and cycloalkyl groups having from 1 to about 20 carbon atoms (e.g., C₁-C₂₀), 1 to 12 carbons (e.g., C₁-C₁₂), 1 to 8 carbon atoms (e.g., C₁-C₈), or, in some embodiments, from 1 to 6 carbon atoms (e.g., C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, carbonyl, halogen, aryl, and heteroaryl groups.

The term “aryl” refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (e.g., C₆-C₁₄) or from 6 to 10 carbon atoms (e.g., C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “halo” or “halogen” refers to one or more halogen atoms, such as fluorine, chlorine, bromine, and iodine.

The term “heteroaryl” represents aromatic ring comprising at least one hetero atom such as N, S, O, or Se. Heteroaryl in the present disclosure may be any hetero aryl. Heteroaryl includes, but is not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, benzimidazolinyl groups, or any combination thereof.

The term “heterocyclyl” refers to substituted or unsubstituted aromatic and non-aromatic ring compounds containing three or more ring members, of which one or more (e.g., 1, 2 or 3) is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (e.g., C₃-C₈), 3 to 6 carbon atoms (e.g., C₃-C₆) or 6 to 8 carbon atoms (e.g., C₆-C₈). A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds, such as in the group 3,6-dihydro-2H-pyran and 3,4-dihydro-2H-pyran respectively, each of which can be substituted.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The term “amine” refers to primary, secondary, and tertiary amines having, e.g., the formula N(group)3 wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The term “compound” as used herein, is meant to include all stereoisomers, geometric isomers, and tautomers of the structures depicted.

The compounds may contain one or more chiral centers, or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, and compositions, methods, uses, and medicaments that include them, may be pure or any of a variety of geometric isomer mixtures. Such mixtures of geometric isomers may include a single configuration at one or more double bonds, while including mixtures of geometry at one or more other double bonds.

The term “solvate” refers to a combination, physical association, and/or solvation of a compound described herein with a solvent molecule such as e.g., a disolvate, monosolvate, or hemisolvate, where the ratio of the solvent molecule to a compound described is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates.

Solvated forms of the compounds can be prepared with a pharmaceutically acceptable solvent. Examples of the solvents include, but are not limited to, water, methanol, and ethanol, and it is intended that the disclosure includes both solvated and unsolvated forms of above-described compounds. One type of solvate is a hydrate. A “hydrate” relates to a particular subgroup of solvates where the solvent molecule is water. Solvates typically can function as pharmacological equivalents. The preparation of solvates is known in the art. For example, M. Caira et al., J. Pharmaceut. Sci., 2004, 93(3): 601-611, describes the preparation of solvates of fluconazole with ethyl acetate and with water. Similar preparation of solvates, hemisolvates, hydrates, and the like are described by E. C. van Tonder et al., AAPS Pharm. Sci. Tech., 2004, 5(1): Article 12, and A.L. Bingham et al, 200, Chem. Commun., 603-604. A typical, non-limiting, process of preparing a solvate would involve dissolving a compound in a desired solvent (organic, water, or a mixture thereof) at temperatures above 20° C. to about 25° C., then cooling the solution at a rate sufficient to form crystals, and isolating the crystals by known methods, e.g., filtration. Analytical techniques, such as infrared spectroscopy, can be used to confirm the presence of the solvate in a crystal of the solvate.

The term “pharmaceutically acceptable salt” refers to salts or zwitterionic forms of compounds described herein.

Examples of the salts of the compounds described herein include, but are not limited to, hydrochloride salt, hydrobromide salt, hydroiodide salt, sulfate salt, bisulfate salt, 2-hydroxyethansulfonate salt, phosphate salt, hydrogen phosphate salt, acetate salt, adipate salt, alginate salt, aspartate salt, benzoate salt, bisulfate salt, butyrate salt, camphorate salt, camphorsulfonate salt, digluconate salt, glycerolphosphate salt, hemisulfate salt, heptanoate salt, hexanoate salt, formate salt, succinate salt, fumarate salt, maleate salt, ascorbate salt, isethionate salt, salicylate salt, methanesulfonate salt, mesitylenesulfonate salt, naphthylenesulfonate salt, nicotinate salt, 2-naphthalenesulfonate salt, oxalate salt, pamoate salt, pectinate salt, persulfate salt, 3-phenylproprionate salt, picrate salt, pivalate salt, propionate salt, trichloroacetate salt, trifluoroacetate salt, phosphate salt, glutamate salt, bicarbonate salt, paratoluenesulfonate salt, undecanoate salt, lactate salt, citrate salt, tartrate salt, gluconate salt, methanesulfonate salt, ethanedisulfonate salt, benzene sulfonate salt, and p-toluenesulfonate salt.

Any reference compounds of the present disclosure appearing herein are intended to include compounds of the present disclosure as well as pharmaceutically acceptable salts or hydrates thereof.

Provided is a pharmaceutical combination for inhibiting metastasis in a patient in need thereof. The pharmaceutical combination comprises (i) a pharmaceutical composition comprising an above-described compound or a pharmaceutically acceptable salt or hydrate thereof, (ii) an additional therapeutic agent, and (iii) optionally at least one pharmaceutically acceptable carrier, excipient, or diluent. In some embodiments, the patient has cancer.

The term “pharmaceutical combination” refers to a pharmaceutical therapy resulting from the mixing or combining of more than one active ingredient. For a combination, the pharmaceutical composition and at least one additional therapeutic agent can be administered to a patient simultaneously or sequentially by the same or different route of administration in a single composition or as two separate compositions to achieve the desired effect. The therapeutic agent can be administered in an amount to provide its desired therapeutic effect. The effective dosage range for each therapeutic agent is well-known in the art, and the therapeutic agent is administered to a patient in need thereof within such established ranges.

The terms “treat,” “treating,” “treated,” and “treatment” (with respect to a disease or condition) are used to describe an approach for obtaining beneficial or desired results, preferably clinical results, and include, but are not limited to, one or more of the following: improving a condition associated with a disease, curing a disease, lessening the severity of a disease, delaying progression of a disease, alleviating one or more symptoms associated with a disease, increasing the quality of life of one suffering from a disease, prolonging survival and/or prophylactic or preventative treatment.

The term “pharmaceutical composition” includes an inhibitory effective amount of one or more compounds for treating a patient, such as a patient with cancer. The composition may include other components and/or ingredients, including, but not limited to, other therapeutically active compounds and/or one or more pharmaceutically acceptable carriers, diluents, excipients, and the like.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffered solutions; and (21) other non-toxic compatible substances employed in pharmaceutical formulations.

The terms “inhibit,” “inhibiting,” “inhibited,” and “inhibition” with regard to inhibiting VAT1 in a patient, such as a patient with cancer, refer to any degree of inhibition of one or more VAT1 in the patient, wherein any degree of inhibition is beneficial to the patient. Desirably, the inhibition of one or more VAT1 inhibits the growth and/or metastasis of cancer in a patient with cancer. The term “inhibitory effect” or “inhibition” refers to the ability of a compound to reduce or block a specific biological or chemical activity, enzyme, or receptor, thereby preventing, suppressing, or altering its normal function.

The term “inhibitory amount” means any amount of a compound or a pharmaceutical composition comprising the compound that is sufficient to achieve an “inhibitor effect” or “inhibition”. The term “effective amount” or “therapeutically effective amount” means the amount of a compound that is effective to treat a disease or a disorder, such as metastatic cancer, at a reasonable benefit/risk ratio. The therapeutically effective amount of such compound will vary depending upon the patient and the disease or disorder being treated, the weight and age of the patient, the severity of the disease or disorder, the manner of administration, and the like, which can readily be determined by one of skill in the art.

The compounds can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the patient including, but not limited to, by oral, intravenous, intratumoral, intramuscular, subcutaneous, transdermal, topical, and like routes of administration.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

Useful dosages of the compounds can be determined by comparing their in vitro activity with their in vivo activity in animal models. Methods of the extrapolation of effective dosages in mice and other animals to human subjects are known in the art. Indeed, the dosage of the compounds can vary significantly depending on the condition of the subject, the age of the subject, the type of disease the subject is experiencing or at risk of experiencing, the particular compounds used, how advanced the pathology is, the route of administration of the compounds and the possibility of co-usage of other therapeutic treatments or additional drugs in combination therapies.

The amount of the composition required for use in inhibition (e.g., the inhibitory effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compound can be administered in an amount ranging from about 1 mg/kg to about 100 mg/kg. In some embodiments, the compound can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

The term “patient” includes human and non-human animals such as companion animals (dogs and cats and the like) and livestock animals. Livestock animals are animals raised for food production. The patient to be treated is preferably a mammal, in particular a human being.

### EXAMPLES

The following examples serve to illustrate the present disclosure. The examples are not intended to limit the scope of the claimed invention in any way.

### Kinetic Solubility of Neocarzilin a and its Derivative Compounds Using Laser Nephelometry

DMSO stock solutions were prepared for each test compound (NCA, NCC, NC-2, NC-6, and NC-9) at a concentration of 100 mM. NC-3 and NC-4 were prepared at 200 mM and 300 mM, respectively. The remaining compounds (NC-5, NC-7, NC-8, and NC-10) were prepared at 500 mM. Compounds were diluted with PBS (pH=7.4) to a final concentration of 0.5% DMSO, vortexed to achieve homogenous mixing, and plated in a VWR 96-flat well plate (Tissue Culture Plate, non-treated, sterilized) in quadruplicate. Compounds were serially diluted with buffer (99.5% PBS pH=7.4, 0.5% DMSO), resulting in 9 different concentrations to fill out the linear ranges of the solubility curve. Every third column in the 96-well plate was filled with buffer to afford proper blanks between the samples. The solubility was then measured using a BMG Labtech 96 laser nephelometer. The 96-well plate was shaken at 100 rpm for 30 seconds prior to running the nephelometry with a laser intensity of 80% and a beam focus of 2.5 mm with a measurement interval time of 0.3 seconds. The solubility was calculated using the equations for the lines of the soluble and insoluble linear ranges in the following equation, with the solubility determined as the intersection between the linear graphs.
For two lines y=mx+b and y′=m′x′+b′, the x value at which the lines intersect is defined by the equation:

$$x = \frac{{b}^{′} - b}{m - {m}^{′}}$$

### UPLC/MS Quantification of the Ratio of Hydration in Trihalomethyl Ketone Covalent Warheads

Stocks of test compounds were prepared in DMSO at 10 mM. 1 μL of the prepared 10 mM stock was diluted with 99 μL PBS (pH=7.4) in Fisher 1.5 mL microcentrifuge tubes (graduated natural) in triplicate to afford a final concentration of 100 μM (1% DMSO in PBS). Compounds were incubated at room temperature for 1 hour to allow for equilibrium to be reached and analyzed using a Waters UPLC™/MS with an ACQUITY™ UPLC™ BEH C18 1.7 μm diameter, 2.1×50 mm column. The mobile phases A and B were H₂O+0.1% formic acid (FA) and acetonitrile+0.1% FA respectively, and samples were run on a gradient of 5-95% mobile phase B over 6 minutes at a flow rate of 0.5 ml min-¹, followed by a gradient of 5-95% mobile phase B from 7 to 7.10 minutes and holding at 5% B until the run ended at 9 minutes. Trihalomethyl ketone and hydration components identification and peak area analysis was carried out using waters_connect™ (UNIFI™) software version 3.0.0.15.

### Determination of IC₅₀ Values by Cell Titer-Glo

MDA-MB-231 cells (RRID: CVCL_0062) were maintained in DMEM (SH30243, Hyclone) with 10% FBS (SH30910.03, Hyclone), lx antibiotic-antimycotic penicillin, streptomycin, and amphotericin B (PSA) and incubated at 37° C. in 5% CO₂ as described (J Pathol 2022, 257 (1), 109-124). Cell viability was determined using the Cell Titer Glo Assay, measuring luminescence with a BioTek Synergy 2 following treatment with NCA or other compounds for 72 hours.

### Scratch/Wound Closure Antimigratory Assay

Scratch assays were performed by seeding cells at a density of 1.5×10⁵ cells/mL. Cells were pretreated with 1.0 μM of NCA or other compounds and incubated for 24 hours. A scratch wound was produced using a sterile pipette tip, and the resulting scratch was imaged at t=0. Media was replaced with 1% FBS DMEM with 1.0 M of NCA or other compounds, and scratch wounds were imaged again at t=24 h. Sixteen independent fields per sample were used and analyzed in ImageJ. The area of each scratch was calculated using the ImageJ software and compared to the area of the scratch at t=0 h. Percent migration was normalized to the DMSO control.

### Transwell Migration Assay

Transwell migration assays were performed with transwell permeable supports (3422, Corning), with 2.5×10⁴ cells suspended in serum-free DMEM. DMEM supplemented with 10% FBS was used as a chemoattractant in the bottom chamber. After 18 hours, non-migrated cells were removed from the inner chamber with a cotton swab, and migrated cells were stained with crystal violet. The resulting stained transwell insert was imaged and quantified by manually counting the number of migrated cells in six independent fields per sample. Each biological replicate was normalized to the corresponding DMSO control which was set to 100% migration.

### Pharmacokinetic Study of NCA, NC-3, and NC-4:

Using C57BL6, female mice, PK evaluation of NCA, NC-3, and NC-4 was performed using the following methods.

A dry stock for each compound was removed from −80° C. storage and thawed on ice at 0° C.

Compounds were first dissolved in DMSO, diluted with HS15 Solutol, followed by PBS to afford the formulated compounds (85% PBS pH=7.4, 10% HS15 Solutol, 5% DMSO) at 3.125 mg/mL concentration. Compounds were drawn up using Medline 1 mL luer slip syringes and animals were dosed via oral gavage using Instech Laboratories 20 gauge oral gavage tips. Animals were euthanized via anesthetization using isoflourane gas, and the secondary method used was cervical dislocation. Terminal blood draw was performed on each animal via cardiac puncture at their respective time points (t=0, 0.25, 0.5, 1, 4 hours). Blood was collected in lithium heparin coated miniCollect blood tubes and stored on ice until plasma would be separated. Plasma was separated from the collected whole blood via centrifugation at 1000 rpm for 5 minutes. The supernatant was carefully pipetted into Fisher 1.5 mL microcentrifuge tubes (graduated natural) and flash-frozen using liquid nitrogen.

### Plasma Sample Preparation for NCA, NC-3, and NC-4 Pharmacokinetics Evaluation

To prepare plasma samples for NCA, 30 μL aliquots of plasma were thawed, and 5 μL of NC-3 (10 ng/μL, 50 ng total) was added to act as the internal standard. Methanol (55 μL) was added to each plasma sample, which was vortexed for 10 minutes. Following vortexing, the samples were centrifuged at 15,000 rpm for 10 minutes, and the supernatant was collected. Samples were either frozen or analyzed immediately following preparation. Preparation of NC-3 and NC-4 plasma samples followed the same procedure, except that NC-10 was utilized as the internal standard (5 μL at 5 ng/μL for a total of 25 ng). A calibration curve was made with a linear range from 1.337 to 1666.7 ng/mL for NCA, NC-3, and NC-4.

### Targeted Analysis of NCA (Internal Standard NC-3) Using Agilent LC/QQQ:

An Agilent 1290 Infinity II liquid chromatography (LC) system coupled to an Agilent 6470 series QQQ mass spectrometer (MS/MS) was used to analyze NCA drug in plasma samples. An Agilent SB-phenyl column (2.1×100 mm, 3.5 um) was used for the analysis of NCA. The mobile phases were composed of (A) H₂O+0.1% formic acid and (B) acetonitrile+0.1% formic acid. The linear LC gradient was as follows: time 0 minutes, 50% B; time 1 minutes, 50% B; time 4 minutes, 100% B; time 6 minutes, 100% B; time 6.1 minutes, 50% B; time 8 minutes, 50% B. The flow rate was 0.4 mL/min. Multiple reaction monitoring was used for MS analysis with the precursor ion (m/z) 321.3 identified for NCA and product ions of 117.1 and 203.3. The internal standard NC-3 had an identified precursor ion 289.3 m/z and product ions of 203.1 and 35.1 m/z. Data were acquired in negative electrospray ionization (ESI) mode. The dwell time was set to 80 msecs, cell accelerator voltage was 4 V, and the fragmenter voltage was 100 V. The jet stream ESI interface had a gas temperature of 325° C., gas flow rate of 8 L/minute, nebulizer pressure of 40 psi, sheath gas temperature of 250° C., sheath gas flow rate of 7 L/minute, capillary voltage of 3500 V in negative mode, and nozzle voltage of 500 V. The AEMV voltage was 400 V. Agilent Masshunter Quantitative analysis software version 10.1 was used for data analysis.

### Targeted Analysis of NC-3 and NC-4 (Internal Standard NC-10) Using Agilent LC/QQQ:

An Agilent 1290 Infinity II liquid chromatography (LC) system coupled to an Agilent 6470 series QQQ mass spectrometer (MS/MS) was used to analyze NC-3/NC-4 in plasma samples. An Agilent eclipse plus C18 (2.1×50, 1.8 um) column was used for the analysis of NC-3/NC-4 drugs. The mobile phases were (A) H₂O+0.1% formic acid and (B) acetonitrile+0.1% formic acid. The linear LC gradient was as follows: time 0 minutes, 25% B; time 1 minute, 25% B; time 11 minutes, 100% B; time 13 minutes, 100% B; time 13.1 minutes, 25% B; time 15 minutes, 25% B. The flow rate was 0.4 mL/min. Multiple reaction monitoring was used for MS analysis. NC-3 was identified with a precursor ion of 289.3 m/z and product ions 203 and 35 m/z. NC-4 was identified with precursor ions of 273.3 m/z and product ions of 203 and 69 m/z. The internal standard for NC-3 and NC-4 was NC-10, which was identified using the precursor ion 323.3 m/z and product ions 203 and 118.9 m/z. Data were acquired in negative electrospray ionization (ESI) mode. The dwell time was set to 80 msecs, cell accelerator voltage was 4 V, and the fragmentor voltage was 100 V. The jet stream ESI interface had a gas temperature of 325° C., gas flow rate of 8 L/minute, nebulizer pressure of 40 psi, sheath gas temperature of 250° C., sheath gas flow rate of 7 L/minute, capillary voltage of 3500 V in negative mode, and nozzle voltage of 500 V. The AEMV voltage was 400 V. Agilent Masshunter Quantitative analysis software version 10.1 was used for data analysis.

### Pharmacokinetic Parameters

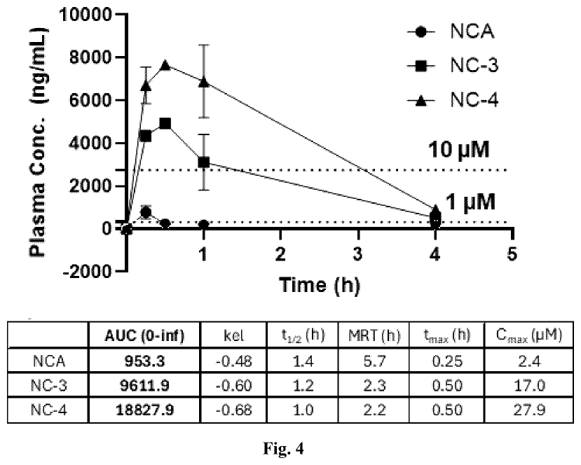

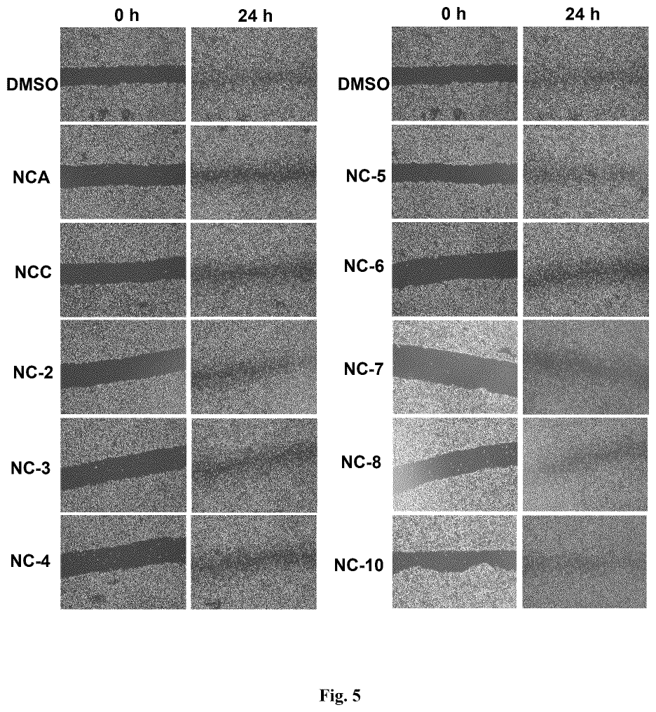

The plasma concentration was plotted versus time in GraphPad Prism (ver. 10.3.0) to afford area under the curve graphs for NCA, NC-3, and NC-4 (FIG. 4). The pharmacokinetic parameters were calculated using a non-compartmental analysis. To determine the elimination rate constant, log 10 of the plasma concentration was plotted against the time and the slope was found for the terminal linear range for each graph. The elimination rate constant was used directly to determine the drug half lives for each compound. The area under the curve (AUC_{0-infinity}) was calculated using the trapezoidal rule, in addition to the area under the moment curve (AUMC_{0-infinity}) by a similar fashion. The ratio of AUMC to AUC was used to determine the mean retention time (MRT), and the maximum concentration (Cₘₐₓ) and time at which the maximum concentration is achieved (tₘₐₓ) was determined. NC-3 and NC-4 were likely still in the distribution phase between 1 and 4 hours. Thus the elimination rate constant is inflated and produced short half-lives and MRT for NC-3 and NC-4 compared to NCA (Clin Transl Sci 2022, 15 (8), 1856).

### Results

### Antiproliferative and Anti-Migratory Activity

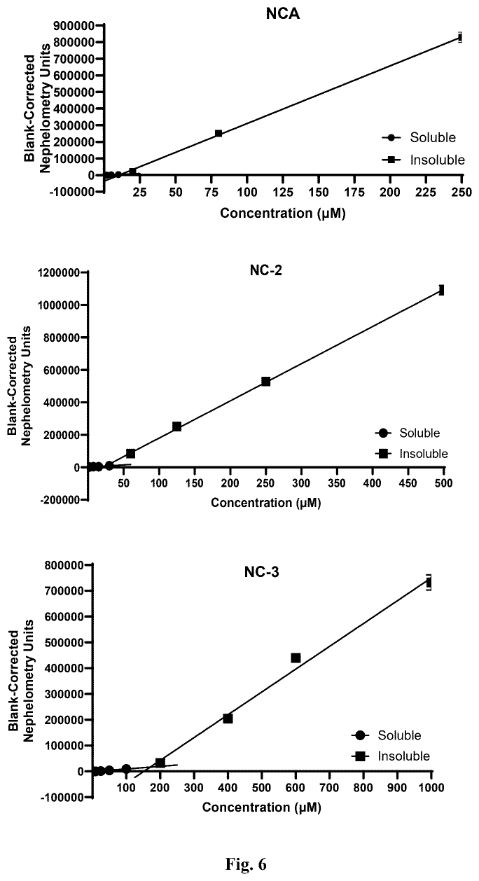

Antimigratory activity was screened via a scratch/wound closure assay (Table 1 and FIG. 5). The triple negative breast cancer cell line MDA-MB-231 was used for initial testing because the VAT1 expression profile is well-understood and NCA has previously shown both antiproliferative and antimigratory activity in this cell line (ACS Cent Sci 2019, 5 (7), 1170-1178). Cells were treated with 1 μM NCA or derivatives, and the ability of the cell monolayer to fill in the scratch over a period of 24 hours was analyzed. Interestingly, the trifluoromethyl ketone derivative NC-4 showed little activity in the scratch/wound closure assay, despite having great solubility and molecular modeling scores. This may be due to the high amount of hydrate present for the trifluoromethyl ketone derivative. In contrast, the chlorodifluoromethyl ketone derivative NC-3 exhibited the best antimigratory activity, suggesting the importance of finding the optimized balance in terms of reducing steric bulk in the active site and limiting the equilibrium of the hydrate (Organofluorine Compounds in Biology and Medicine 2015, 29-57). Compound NC-5, which has a relatively unreactive methyl group in place of the trihalomethyl group, had no activity.

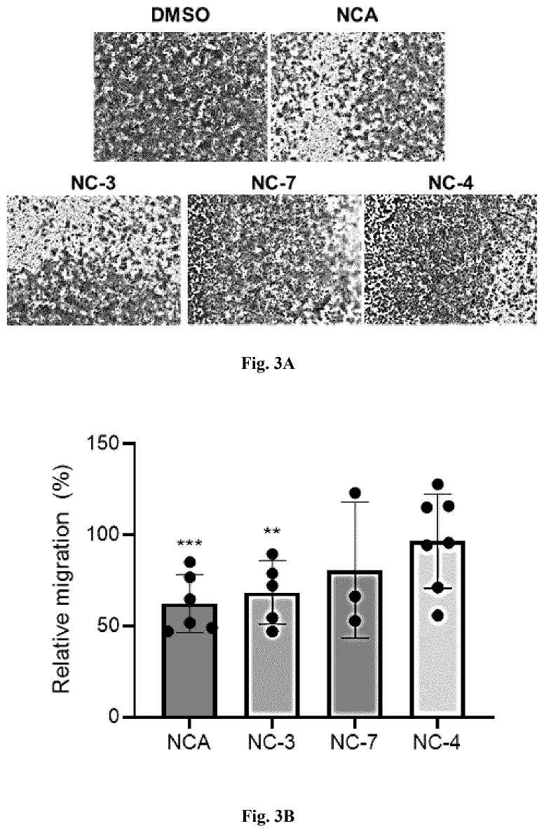

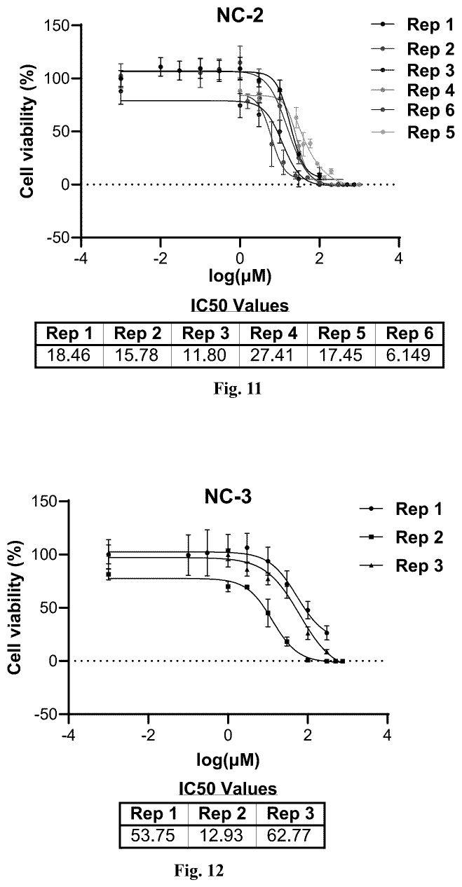

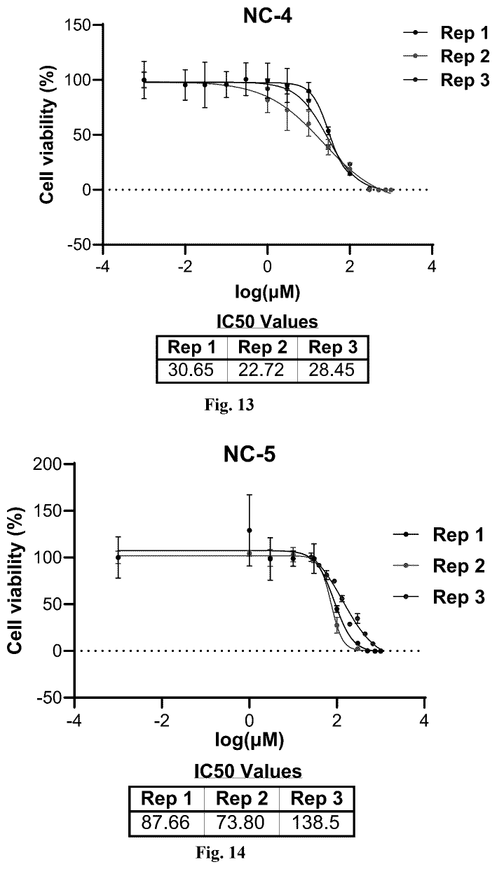

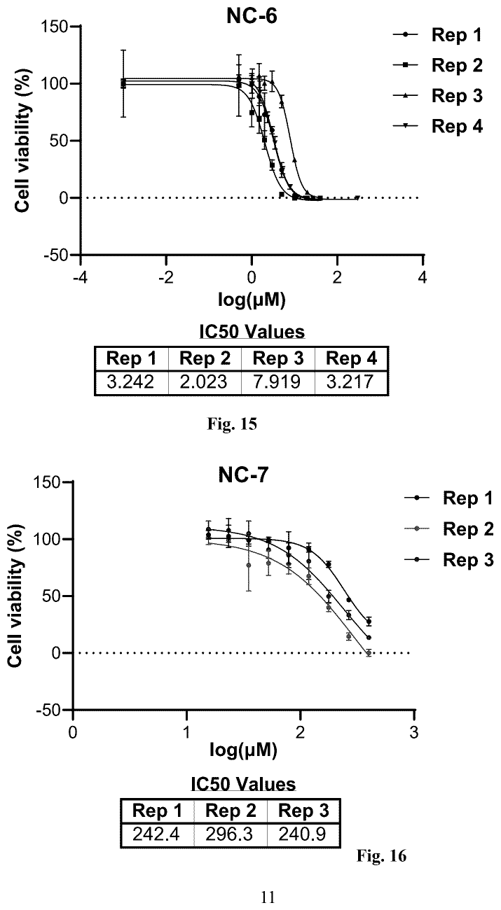

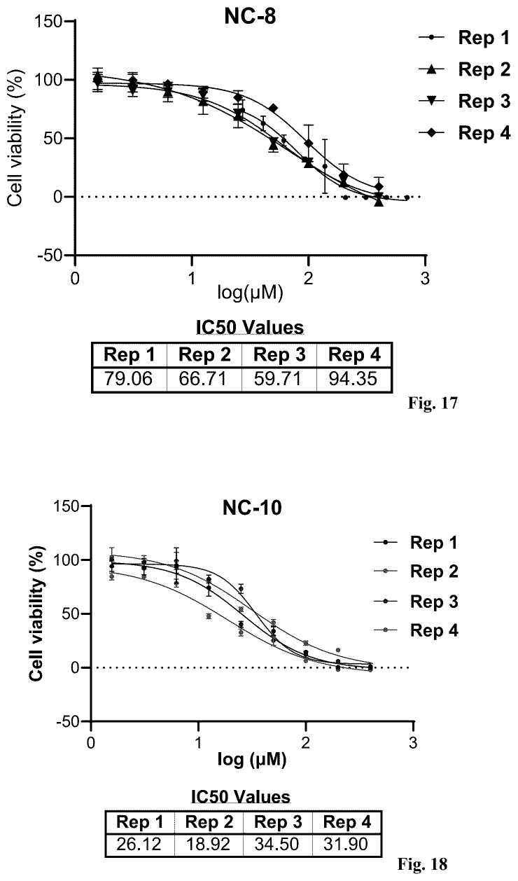

The antiproliferative activities of the compounds were then measured using the CellTiter Glo viability assay (Table 1 and FIGS. 10-18). The parent NCA exhibits rather potent antiproliferative activity (IC₅₀<1 μM) in a variety of healthy and cancerous cell lines, despite genetic knockdown models for VAT1 showing no effect on cell viability. This suggests that NCA exhibits polypharmacology, and thus it was speculated that the derivatives with attenuated warhead reactivity would have reduced affinity for the targets that caused the antiproliferative activity. It was reported that small changes to the scaffold, such as the alkyne derivative used for chemoproteomics, resulted in a 10-fold reduction of the antiproliferative activity but retention of the antimigratory activity against VAT1. The compounds were screened against the MDA-MB-231 cells and found that the removal of one of the chlorines of NCA to make the natural product NCC resulted in a reduction in the antiproliferative and antimigratory activity, corroborating the results of known studies (ACS Cent Sci 2019, 5 (7), 1170-1178). The truncated alkene derivative NC-5 exhibited a modest reduction in the antiproliferative activity (IC₅₀=3.8 μM), although the elongated derivative NC-9 exhibited even greater potency than the parent NCA (700 nM), suggesting the length and hydrophobic character were important for the antiproliferative activity. The greatest effect on antiproliferative activity was observed for the replacement of the chlorines with fluorines. The addition of fluorines to the warhead resulted in a stepwise decrease in cytotoxicity, for both the normal length NC-2 to NC-4 derivatives and the truncated NC-6 and NC-7 derivatives (Table 1 and FIGS. 10-18). Interestingly, despite a potent reduction in the antiproliferative activity, compounds NC-2 to NC-4 retained their antimigratory activity in the scratch/wound closure assay at 1 μM. This encouraging result suggests that the warhead plays a significant role in the antiproliferative to antimigratory bioactivity ratio, leading us to compare them using a selectivity index (i.e. the ratio of antimigratory activity at 1 μM to the antiproliferative IC₅₀). Compounds that exhibited a selectivity index greater than ten were selected for confirmation of the antimigratory activity using a transwell migration assay (FIGS. 3A and 3B). Although the truncated alkene derivatives NC-7 to NC-8 exhibited the greatest improvements to solubility and the highest selectivity index, they failed to exhibit potent reduction of migration in the transwell migration assay, leaving compounds NC-3 and NC-4 as the most potent derivatives in the assays tested. NC-4 exhibited a lower reduction in migration than NC-3, suggesting the high level of hydration of the trifluoromethylketone warhead can impede inhibition of VAT1, resulting in lower reductions in migration than the chlorodifluoromethylketone counterpart of NC-3. To further evaluate the effect of hydration on binding VAT1, the hydrated versions of NCA, NC-3, and NC-4 were modelled noncovalently in the putative substrate binding pocket of VAT1 (PDB: 6LHR). Since the covalent chemistry would not be possible with hydrate, covalent docking was omitted. From the docking study, it was found in all cases that the hydrated compounds had considerably worse docking scores and output poses than their ketone counterparts. This further suggests that high levels of hydration may negatively affect inhibitor binding to VAT1.

### Pharmacokinetics Analysis

To evaluate the pharmacokinetics, NCA and the compounds NC-3 or NC-4 were administered to mice at 25 mg/kg via oral gavage (per os, p.o.) administration. The formulation of NCA was a cloudy homogeneous suspension at 25 mg/kg (85% PBS pH=7.4, 10% Solutol, 5% DMSO), but NC-3 and NC-4 were completely clear solutions, highlighting their increased solubility. Following compound administration, the plasma concentration was monitored over a period of four hours. Due to the poor solubility and oxidative liability of the polyene core of NCA, it was expected that its clearance would be quite rapid, hence the reason for analysis being terminated after 4 hours. Additionally, in predicting the metabolism of NCA using SwissADME calculations of drug-like properties, it was identified as a substrate for both CYP2C19 and CYP2C9. NCA exhibited a very short half-life and was largely cleared from systemic circulation after the first hour (FIG. 4). The fluorinated compounds maintained a much longer residence time in circulation, with relatively high concentrations of compound still remaining at the four-hour time point when the experiment was terminated. The compounds NC-3 and NC-4 both exhibited substantially increased AUC(0-infinity) in comparison to NCA. This highlights the increased concentration of the drug that was entering circulation and was available for distribution, compared to NCA and was reflected by a longer tₘₐₓ and a higher Cₘₐₓ for NC-3 and NC-4. However, while NCA was clearly in the elimination phase by t=4 hours, NC-3 and NC-4 were still likely in the distribution and post-absorption phases and were not exhibiting a linear elimination range from which to determine an accurate elimination rate constant. This conflates the data leading to lower half-lives and mean retention times, confounding data comparison with NCA. However, given the significantly improved AUC for these compounds, it was clear that they are being absorbed and entering systemic circulation at a much higher rate than the parent NCA. This allowed to maintain therapeutically relevant concentrations (>1 μM) even at the terminal 4-hour time point for both NC-3 and NC-4, while NCA dropped below this value after the first 15 minutes. This highlights the utility of improving the aqueous solubility to improve the pharmacokinetics of this class of compounds. It also corroborates previous results, highlighting that more soluble novobiocin derivatives exhibited slower clearance and improved bioavailability than the more poorly soluble initial hits (Bioorg Med Chem 2023, 92, 117381). The results were similar in a covalent inhibitor system.

### General Information

### Materials and Methods

All reagents and solvents were purchased reagent grade or higher from commercial vendors (Sigma-Aldrich, Thermo Fisher Scientific Inc., Oakwood Chemical, Alfa Aesar, Acros Chemicals) and were used without further purification. All reactions were conducted using flame-dried glassware. Reactions containing oxygen or water-sensitive reagents were carried under a nitrogen atmosphere. For reaction monitoring, analytical thin-layer chromatography (TLC) was carried out on silica-gel 60 F254 plates, using short wave UV light (λ=254 nm) or KMnO₄-stain (1.50 g KMnO₄, 10.0 g K₂CO₃, 1.25 mL NaOH aq (10 wt-%), 200 mL ddH₂O) to visualize reaction components.

¹H-NMR experiments were recorded on NMR systems at room temperature with CDCl₃ as the solvent and referenced to the residual proton signal of the corresponding deuterated solvent (CDCl₃: δ=7.26 ppm). Chemical shifts are reported in parts per million (ppm). Coupling constants (J) are reported in hertz (Hz) and for the assignment of multiplicity to the signals the following abbreviations were used: s=singlet, d=doublet, t=triplet, q=quartet, m=multiplet or unresolved.

Unless otherwise noted, ultra-high performance liquid chromatography mass spectrometry (UPLC-MS) analyses were performed using a Waters™ ACQUITY™ UPLC™ in line with an ACQUITY RDa Detector and an Acquity UPLC BEH C18 1.7 μm diameter, 2.1×50 mm column. Waters_connect (Unifi) software v.3.0.0.15 was used in the analyses of the spectra.

### EXAMPLES

### Example 1

### Ethyl (E)-4-(diethoxyphosphoryl)-but-2-enoate

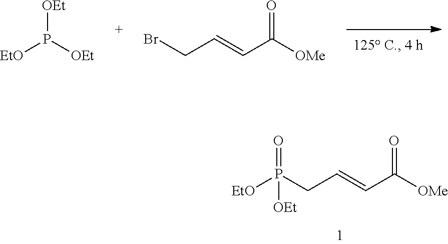 [SMILES: CCOP(OCC)OCC ; COC(=O)/C=C/CBr ; CCOP(=O)(C/C=C/C(=O)OC)OCC]

To a stirred solution of methyl-4-bromocrotonate (5 g, 27.93 mmol) was added triethyl phosphite (5.1 g, 30.7 mmol). The mixture was stirred neat, warmed to 125° C., and refluxed for 4 hours. The crude oil was purified via vacuum distillation (115° C., 0.04 mbar) to afford 6.5 g of the phosphonate 1 (27.5 mmol, quant.) as a viscous yellow oil. ¹H NMR (400 MHz, CDCl₃) δ 6.87 (h, J=7.6 Hz, 1H), 5.98 (dd, J=15.6, 4.9 Hz, 1H), 4.15-4.07 (m, 4H), 3.73 (s, 3H), 2.77 (d, J=7.6 Hz, 1H), 2.71 (d, J=7.6 Hz, 1H), 1.32 (t, J=7.0 Hz, 6H). Spectra is in accordance with previously reported data (ACS Cent Sci, 2019, 5 (7), 1170-1178; Tetrahedron Lett, 1992, 33 (49), 7551-7552).

### Example 2

### Ethyl-2-(diethoxyphosphoryl)acetate

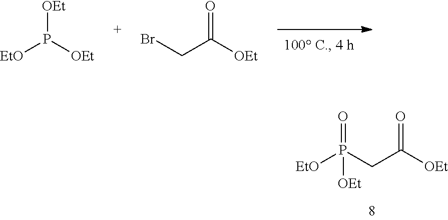 [SMILES: CCOP(OCC)OCC ; CCOC(=O)CBr ; CCOC(=O)CP(=O)(OCC)OCC]

To a stirred solution of ethyl bromoacetate (5 g, 29.94 mmol) was added triethyl phosphite (5.5 g, 32.9 mmol). The mixture was stirred neat, warmed to 100° C., and refluxed for 4 hours. The crude oil was purified via vacuum distillation (105° C., 0.04 mbar) to afford 6.5 g of the phosphonate 8 (30 mmol, quant.) as a viscous clear oil. ¹H NMR (400 MHz, CDCl₃) δ 4.18 (h, J=7.2 Hz, 6H), 2.98 (s, 1H), 2.92 (s, 1H), 1.34 (t, J=7.1 Hz, 6H), 1.28 (t, J=7.0 Hz, 3H). Spectra is in accordance with previously published data (European Journal of Organic Chemistry, 2002(17), 3015-3023).

### Example 3

### Methyl (S,2E,4E)-6-methylocta-2,4-dienoate

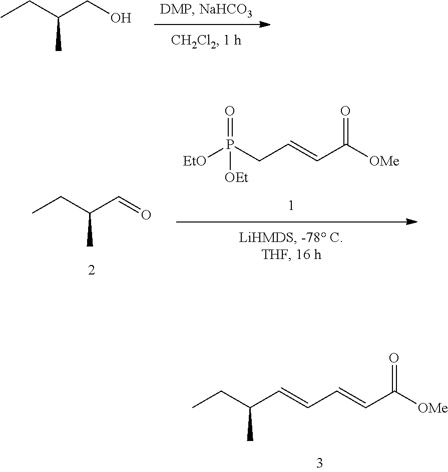

The Dess-Martin Periodinane reagent (DMP, 6 g, 14.2 mmol) and NaHCO₃ (1.9 g, 22.7 mmol) were dissolved in 15 mL of dry CH₂Cl₂ in a flame-dried flask and cooled to 0° C. in an ice bath. S-2-methyl-butan-1-ol (1 g, 11.34 mmol) was added slowly and stirred at 0° C. for 45 minutes. The reaction mixture was allowed to warm to room temperature and was diluted with diethyl ether (25 mL). The reaction was quenched by the slow addition of NaOH (3.75 M, 20 mL). The resulting suspension was allowed to stir 15 minutes until the mixture became clear, upon which the organic layer was separated, and the aqueous was further washed with 10 mL of diethyl ether twice. The organics were combined and dried with Na₂SO₄, filtered, and the ether was removed under a stream of N₂ to retain the volatile aldehyde. In parallel, phosphonate 1 (3.75 g, 15.90 mmol) was dissolved in anhydrous THE (10 mL) and cooled to −78° C. LiHMDS (1M in THF, 17.01 mL, 17.01 mmol) was added slowly, and the reaction was stirred for 1 hour at −78° C. followed by the addition of aldehyde. The reaction was stirred for 1 hour, upon which it was allowed to warm to −40° C. and stir a further 4 hours. The reaction was then diluted with diethyl ether (50 mL) and allowed to warm to 0° C. and was quenched by the addition of saturated aq. NH₄Cl. The organics were washed with saturated aq. NH₄Cl, dried with Na₂SO₄, filtered, and concentrated. The crude product was purified via flash chromatography (Rf=0.25, 3% EtOAc/Hex, [UV, KMNO₄]) to afford the product 3 (1.33 g, 70% over two steps) as a yellow oil. ¹H NMR (400 MHz, CDCl₃) δ (ppm)=7.26 (dd, J=15.7, 10.6 Hz, 1H), 6.19-5.96 (m, 2H), 5.80 (d, J=15.4 Hz, 1H), 3.73 (s, 3H), 2.16 (hept, 1H), 1.37 (d, J=7.5 Hz, 2H), 1.03 (d, J=6.7 Hz, 3H), 0.86 (t, J=7.1 Hz, 3H). Spectra is in accordance with previously published data (ACS Cent Sci, 2019, 5 (7), 1170-1178; Tetrahedron Lett, 1992, 33 (49), 7551-7552).

### Example 4

### (S,2E,4E)-6-methylocta-2,4-dien-1-ol

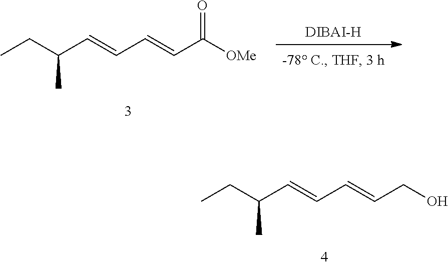

The methyl ester 3 (1 g, 5.94 mmol) was dissolved in anhydrous THF (10 mL) and cooled to −78° C. DIBAL-H (1M in hexane, 14.9 mL, 14.9 mmol) was added slowly, and the reaction was stirred at −78° C. for 3 hours. The reaction was allowed to warm to 0° C. and diluted with diethyl ether (40 mL). The reaction was quenched by the addition of saturated aq. potassium sodium tartrate (50 mL) and was stirred for 1 hour. The aqueous was washed with diethyl ether three times (50 mL), and the organics were combined, dried with Na₂SO₄, filtered, and concentrated to afford the alcohol 4. The resulting colorless oil (0.82 g, quant.) was sufficiently pure to use without purification. ¹H NMR (400 MHz, CDCl₃) δ (ppm)=6.28-6.17 (m, 1H), 6.01 (dd, J=15.2, 10.4 Hz, 1H), 5.74 (dt, J=15.2, 6.1 Hz, 1H), 5.59 (dd, J=15.2, 7.8 Hz, 1H), 4.20-4.12 (m, 2H), 2.07 (hept, J=6.9 Hz, 1H), 1.38-1.29 (m, 2H), 0.99 (d, J=6.7 Hz, 3H), 0.85 (t, J=7.4 Hz, 3H). Spectra is in accordance with previously published data (ACS Cent Sci, 2019, 5 (7), 1170-1178).

### Example 5

### (S,3E,5E,7E)-9-methylundeca-3,5,7-trien-2-one

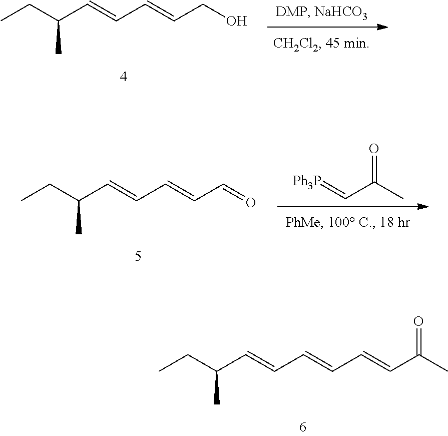

DMP (2.53 g, 5.98 mmol) and NaHCO₃ (1 g, 11.96 mmol) were dissolved in anhydrous CH₂Cl₂ (20 mL) in a flame-dried flask. The mixture was cooled to 0° C., and alcohol 4 (0.67 g, 4.78 mmol) was added. The reaction was allowed to stir for 45 minutes before being diluted with diethyl ether (30 mL) quenched with the addition of NaOH (3.75 M, 20 mL) and stirred for 10 minutes. The organics were washed with NaOH (1 M, 20 mL), dried with Na₂SO₄, filtered, and concentrated to a colorless oil. The aldehyde 5 was dissolved in anhydrous toluene (15 mL) and added to 1-(triphenylphosphoranylidene)-2-propanone (3.04 g, 9.56 mmol) in a flame-dried flask. The reaction was heated near reflux and stirred for 18 hours at 100° C. The reaction was then slowly cooled to 0° C. and cold hexanes were added to precipitate the triphenyl phosphine oxide (TPPO) byproduct. The TPPO was filtered off, the filtrate evaporated, and the precipitation was repeated twice, or until the majority of the TPPO was removed. The crude product was purified via flash chromatography (Rf=0.32 (10% EtOAc in Hexanes) [UV, KMnO₄]) to afford the ketone 6 (0.44 g, 2.5 mmol, 52% over two steps) as a pale-yellow oil. ¹H NMR (400 MHz, CDCl₃) δ (ppm)=7.14 (dd, J=15.6, 11.1 Hz, 1H), 6.58 (dd, J=14.9, 10.7 Hz, 1H), 6.24 (dd, J=14.9, 11.1 Hz, 1H), 6.17-6.07 (m, 2H), 5.84 (dd, J=15.2, 7.8 Hz, 1H), 2.27 (s, 3H), 2.15 (p, J=7.0 Hz, 1H), 1.45-1.30 (m, J=7.3, 6.7 Hz, 2H), 1.02 (d, J=6.7 Hz, 3H), 0.87 (t, J=7.4 Hz, 3H). Spectra is in accordance with previously published data (ACS Cent Sci, 2019, 5 (7), 1170-1178; Tetrahedron Lett, 1992, 33 (49), 7551-7552).

### Example 6

### Ethyl (S,E)-4-methylhex-2-enoate

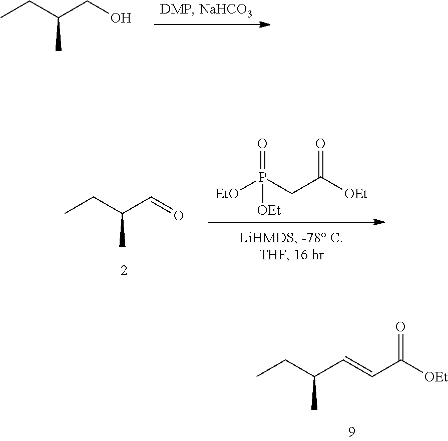

Ester 9 was synthesized according to a similar procedure as 3. Briefly, DMP (6 g, 14.2 mmol) and NaHCO₃ (1.9 g, 22.7 mmol) were dissolved in dry DCM (20 mL) in a flame-dried round bottom flask and cooled to 0° C. After stirring for 5 minutes, S-2-methylbutanol was added slowly and the reaction stirred for 1 hour and allowed to warm to room temperature. In parallel, ethyl-2-(diethoxyphosphoryl)-acetate 8 (3.6 g, 16 mmol) was dissolved in dry THE (8 mL) in a flame dried round bottom flask and cooled to −78° C. LiHMDS (17 mL, 1 M in THF, 17 mmol) was added slowly and allowed to stir for 1 hour. To quench the oxidation, diethyl ether (30 mL) was added followed by NaOH (2 M, 50 mL). This was allowed to stir 15 minutes until the aqueous layer became clear and the layers were separated. The aqueous layer was washed with diethyl ether (30 mL total), and the organics were combined, dried with anhydrous NaSO₄, and concentrated under a stream of N₂ to afford a slightly volatile aldehyde 2. This aldehyde was added to the phosphonate 8 at −78° C. and stirred for 1 hour. The reaction was then allowed to warm to −20° C. and stir overnight. To quench, the reaction was allowed to warm to room temperature and diluted with diethyl ether (100 mL) and washed three times with sat. NH₄Cl, followed by brine. The organics were then dried with Na₂SO₄ and concentrated in vacuo to afford the ester 9 as a colorless oil. ¹H NMR (400 MHz, CDCl₃) δ 6.86 (dd, J=15.7, 7.8 Hz, 1H), 5.77 (d, J=15.7 Hz, 1H), 4.18 (q, J=7.0 Hz, 2H), 2.21 (hept, J=6.9 Hz, 1H), 1.40 (p, J=7.3 Hz, 2H), 1.29 (t, J=7.1 Hz, 3H), 1.05 (d, J=6.6 Hz, 3H), 0.88 (t, J=7.4 Hz, 3H). Spectra is in accordance with previously published data (Org Biomol Chem 2003, 1 (19), 3362-3376).

### Example 7

### (S,E)-4-Methylhex-2-en-1-ol

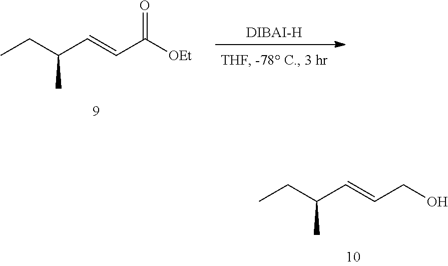

Alcohol 10 was synthesized using a modified procedure from alcohol 4. Briefly, 9 (0.44 g, 2.8 mmol) was dissolved in dry DCM (5 mL) and cooled to −78° C. DIBAl—H (7 mL, 7 mmol, 1M in hexanes) was added slowly, and the reaction stirred for 3 hours at −78° C. Upon completion the reaction was allowed to warm to room temperature, diluted with 50 mL DCM, and was quenched by slow addition of MeOH (until bubbling ceases), followed by water and 1M HCl (50 mL). The reaction was transferred to a separatory funnel, the organic layer was collected, and the aqueous was extracted with DCM (3×50 mL). The organics were combined, dried with Na₂SO₄, and concentrated to afford the alcohol 10 as a colorless oil (0.3 g, 94%). ¹H NMR (400 MHz, CDCl₃) δ 5.65-5.52 (m, 2H), 4.09 (d, J=4.6 Hz, 2H), 2.04 (hept, J=6.7 Hz, 1H), 1.31 (p, J=7.0 Hz, 3H), 0.99 (d, J=6.8 Hz, 3H), 0.86 (t, J=7.4 Hz, 3H). Spectra is in accordance with previously published data (Org Lett 2001, 3 (15), 2289-2291).

### Example 8

### (S,3E,5E)-7-methylnona-3,5-dien-2-one

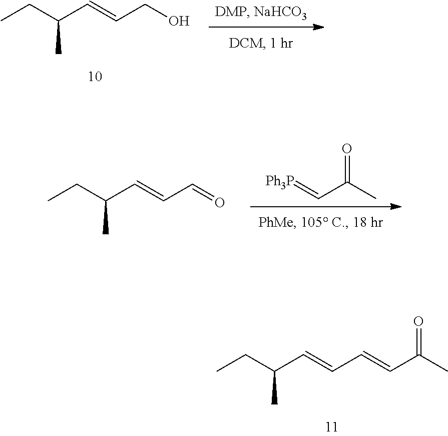

Ketone 11 was synthesized in a similar fashion as ketone 6. Briefly, DMP (3 g, 7.1 mmol) and NaHCO₃ (0.96 g, 11.4 mmol) were dissolved in dry DCM (12 mL) and cooled to 0° C. After stirring for 5 minutes, alcohol 10 (0.65 g, 5.7 mmol) was added and the reaction stirred for 1 hour, allowing it to warm to room temperature during the duration. The reaction was diluted with diethyl ether (100 mL) and quenched with aq. NaOH (2M, 50 mL). This stirred for 15 minutes until the two layers were clear and colorless. The DCM layer was separated and the aqueous was extracted with DCM (3×50 mL). The organics were combined, dried with Na₂SO₄, and concentrated in vacuo to afford the aldehyde as a slightly yellow oil. The aldehyde was dissolved in dry toluene (20 mL) in a flame-dried flask. Triphenylphosphoranylidene propan-2-one (3.6 g, 11.4 mmol) was then added and the reaction was heated to 105° C. and stirred for 18 hours. Upon completion, the reaction was cooled to 0° C. and the TPPO byproduct was precipitated by addition of cold hexanes, filtered, and concentrated. This was repeated twice, or until TPPO was sufficiently removed such that the product could then be purified and isolated via flash chromatography (Rf=0.17, 95:5 Hex/EtOAc) to afford the ketone 11 as an orange-yellow oil (0.51 g, 3.4 mmol, 60% over 2 steps).

¹H NMR (400 MHz, CDCl₃) δ 7.10 (dd, J=15.6, 10.3 Hz, 1H), 6.16 (dd, J=15.3, 10.2 Hz, 1H), 6.11-6.02 (m, 2H), 2.26 (s, 3H), 2.19 (p, J=6.9 Hz, 1H), 1.37 (hept, J=7.2 Hz, 2H), 1.03 (d, J=6.7 Hz, 3H), 0.87 (t, J=7.4 Hz, 3H). Spectra is in accordance with previously published data (ACS Cent Sci, 2019, 5 (7), 1170-1178).

### Example 9

### Ethyl (S,2E,4E,6E)-8-methyldeca-2,4,6-trienoate

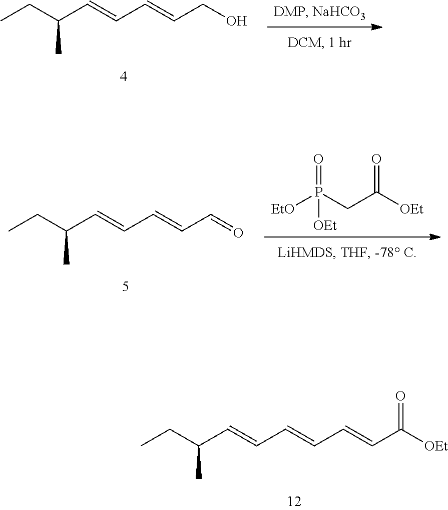

DMP (1.5 g, 3.6 mmol) and NaHCO₃ (0.06 g, 0.7 mmol) were dissolved in dry THF (20 mL) and cooled to 0° C. After stirring 5 minutes, alcohol 4 (0.4 g, 2.8 mmol) was added and the reaction was allowed to warm to room temperature and stir for 1 hour. In parallel, phosphonate 8 (0.96 g, 4.3 mmol) was dissolved in anhydrous THF (20 mL) and cooled to −78° C. LiHMDS (1M in THF, 4.3 mL, 4.3 mmol) was added slowly and stirred for 1 hour. Upon completion of the DMP oxidation, the reaction was diluted with diethyl ether (50 mL) and quenched by addition of aq. NaOH (2M, 50 mL) and stirred 15 minutes until the aqueous layer turned clear. The organic layer was separated and washed further with aq. NaOH (2M, 25 mL). The organic layer was collected, dried with Na₂SO₄ and concentrated under vacuum to afford the aldehyde 5. The aldehyde was then added directly to the flask containing the generated phosphonate ylide at −78° C. and stirred 1 hour before allowing to warm to −20° C. and stirring overnight. The reaction was then allowed to warm to rt, diluted with 100 mL ether, and quenched by addition of sat. aq. NH₄Cl (3×100 mL) followed by a brine wash. The remaining organic layer was collected, dried with Na₂SO₄ and concentrated under vacuum and purified using flash chromatography (Rf=0.34, 95:5 Hex/EtOAc) to afford the ester 12 as a yellow oil (0.29 g, 48% over 2 steps). ¹H NMR (400 MHz, CDCl₃) δ 7.30 (dd, J=15.3, 11.2 Hz, 1H), 6.52 (dd, J=14.9, 10.7 Hz, 1H), 6.22 (dd, J=14.9, 11.3 Hz, 1H), 6.10 (dd, J=15.2, 10.7 Hz, 1H), 5.94-5.70 (m, 2H), 4.20 (q, J=7.1 Hz, 2H), 2.13 (hept, J=7.0 Hz, 1H), 1.35 (p, J=7.4 Hz, 2H), 1.29 (t, J=7.1 Hz, 3H), 1.01 (d, J=6.7 Hz, 3H), 0.86 (t, J=7.4 Hz, 3H). Spectra is in accordance with previously published data (ACS Cent Sci, 2019, 5 (7), 1170-1178).

### Example 10

### (S,2E,4E,6E)-8-methyldeca-2,4,6-trien-1-ol

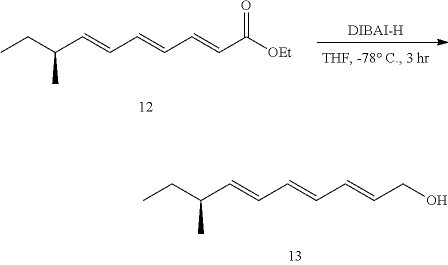

Ester 12 (0.29 g, 1.4 mmol) was dissolved in anhydrous THF (10 mL) and cooled to −78° C. DIBAl-H (1M in hexanes, 3.44 mL, 3.44 mmol) was added slowly and the reaction stirred at −78° C. for 3 hours. Upon completion, the reaction was allowed to warm to room temperature, diluted with diethyl ether (40 mL), and quenched with sat. aq. sodium potassium tartrate (50 mL) and stirred for 30 minutes. The organic layer was collected and transferred to a separatory funnel and further washed with sat. aq. sodium potassium tartrate (3×40 mL) followed by brine (50 mL). The resulting organics were collected, dried with Na₂SO₄ and concentrated under vacuum to afford the crude alcohol 13 in sufficient purity to use without further purification as a colorless oil (0.22 g, quant.). ¹H NMR (400 MHz, CDCl₃) δ 6.32-6.07 (m, 3H), 6.03 (dd, J=15.1, 10.1 Hz, 1H), 5.80 (dt, J=15.0, 6.1 Hz, 1H), 5.61 (dd, J=15.1, 7.8 Hz, 1H), 4.18 (d, J=6.0 Hz, 2H), 2.09 (hept, J=6.9 Hz, 1H), 1.33 (p, J=7.3 Hz, 2H), 0.99 (d, J=6.7 Hz, 3H), 0.85 (t, J=7.4 Hz, 3H).

### Example 11

### (S,3E,5E,7E,9E)-11-methyltrideca-3,5,7,9-tetraen-2-one

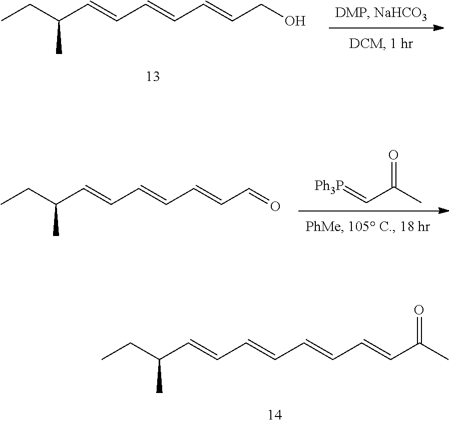

Ketone 14 was synthesized in a similar fashion as ketone 6. Briefly, DMP (0.73 g, 1.7 mmol) and NaHCO₃ (29 mg, 0.34 mmol) were dissolved in dry DCM (8 mL) and cooled to 0° C. After stirring for 5 minutes, 13 (0.23 g, 1.4 mmol) was added and the reaction stirred for 1 hour, allowing it to warm to room temperature during the duration. The reaction was diluted with diethyl ether (40 mL) and quenched with aq. NaOH (2M, 40 mL). This stirred for 15 minutes until the two layers were clear and colorless. The organic layer was separated and the aqueous was extracted with DCM (3×30 mL). The organics were combined, dried with Na₂SO₄ and concentrated in vacuo to afford the aldehyde as a yellow oil. The aldehyde was dissolved in dry toluene (20 mL) in a flame-dried flask. Triphenylphosphoranylidene propan-2-one (0.87 g, 2.7 mmol) was then added and the reaction was heated to 105° C. and stirred for 18 hours. Upon completion, the reaction was cooled to 0° C. and the TPPO byproduct was precipitated by the addition of cold hexanes, filtered, and concentrated. This was repeated twice, or until TPPO was sufficiently removed such that the product could then be purified and isolated via flash chromatography (Rf=0.34, 95:5 Hex/EtOAc) to afford the ketone 14 as an orange oil (102 mg, 37% over 2 steps). ¹H NMR (400 MHz, CDCl₃) δ 7.16 (dd, J=15.2, 11.2 Hz, 1H), 6.62 (dd, J=14.8, 11.0 Hz, 1H), 6.40 (dd, J=14.9, 10.6 Hz, 1H), 6.30 (dd, J=14.8, 11.1 Hz, 1H), 6.26-6.18 (m, 1H), 6.16-6.05 (m, 2H), 5.76 (dd, J=15.1, 7.9 Hz, 1H), 2.27 (s, 3H), 2.13 (hept, J=7.0 Hz, 1H), 1.38-1.32 (m, 2H), 1.01 (d, J=6.7 Hz, 3H), 0.87 (d, J=7.4 Hz, 3H). Spectra is in accordance with previously published data (ACS Cent Sci, 2019, 5 (7), 1170-1178).

### Example 12

### (S,3Z,5E,7E,9E)-1,1,1-trichloro-4-hydroxy-11-methyltrideca-3,5,7,9-tetraen-2-one (Neocarzili A)0

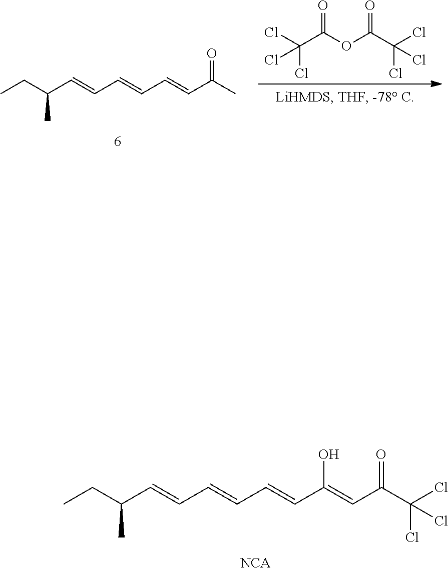 [SMILES: CC[C@H](C)/C=C/C=C/C=C/C(C)=O ; O=C(OC(=O)C(Cl)(Cl)Cl)C(Cl)(Cl)Cl ; "6" ; CC[C@H](C)/C=C/C=C/C=C/C(O)=C/C(=O)C(Cl)(Cl)Cl ; "NCA"]

Ketone 6 (0.10 g, 0.56 mmol) was dissolved in anhydrous THF (7 mL) in a flame dried flask and cooled to −78° C. LiHMDS (1M in THF, 0.62 mL, 0.62 mmol) was added slowly, and the reaction was allowed to stir at −78° C. for 1 hour. Trichloroacetic anhydride (0.21 mL, 1.12 mmol, 2 equiv.) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated to a bright yellow oil. The crude product was purified via flash chromatography (Rf=0.38, 5% EtOAc in Hexanes, [UV, KMnO₄]) to afford Neocarzilin A (135 mg, 0.42 mmol, 75%) as an orange oil. Spectra is in accordance with previously published data (ACS Cent Sci, 2019, 5 (7), 1170-1178; Tetrahedron Lett, 1992, 33 (49), 7551-7552).

¹H NMR (400 MHz, CDCl₃) δ (ppm)=7.37 (ddd, J=14.9, 11.5, 0.5 Hz, 1H), 6.62 (dd, J=14.8, 10.7 Hz, 1H), 6.27 (dd, J=14.8, 11.3 Hz, 1H), 6.21-6.08 (m, 2H), 6.00 (d, J=15.0 Hz, 1H), 5.89 (dd, J=15.2, 7.8 Hz, 1H), 2.17 (p, J=6.9 Hz, 1H), 1.45-1.31 (m, 2H), 1.03 (d, J=6.7 Hz, 3H), 0.87 (t, J=7.4 Hz, 3H). ¹³C NMR (CDCl₃) δ (ppm)=186.0, 177.9, 147.6, 142.9, 142.7, 128.3, 128.3, 123.2, 95.0, 93.0, 38.8, 29.4, 19.6, 11.6. [α]²²D=49.6 (c=1.0, CHCl₃). Mass spec: expected neutral mass for C₁₄H₁₇Cl₃O₂ (Da): 322.02941, observed neutral mass (Da): 322.0303, mass error (ppm) 2.8. UPLC Trace: Obtained using mobile phases of H₂O+0.1% formic acid (A) and acetonitrile+0.1% formic acid (B). Samples were eluted using a gradient mode with mobile phase B ranging from 5% to 95% over 8 minutes at a flow rate of 0.5 mL/min. Then the 95% B from 9 to 9.1 minutes was on a gradient to 5% B until the end at 14 minutes. The purity was determined to be >99%.

### Example 13

### (S,3Z,5E,7E,9E)-1,1,1-trifluoro-4-hydroxy-11-methyltrideca-3,5,7,9-tetraene-2-one

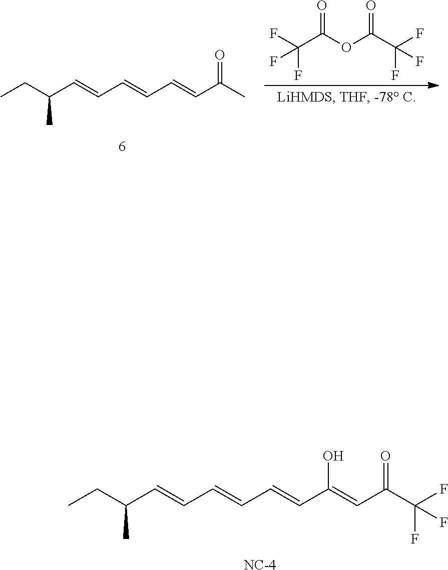 [SMILES: CC[C@H](C)/C=C/C=C/C=C/C(C)=O ; O=C(OC(=O)C(F)(F)F)C(F)(F)F ; "6" ; CC[C@H](C)/C=C/C=C/C=C/C(O)=C/C(=O)C(F)(F)F ; "NC-4"]

Ketone 6 (0.10 g, 0.56 mmol) was dissolved in anhydrous THF (7 mL) and cooled to −78° C. LiHMDS (1M in THF, 0.55 mL, 0.55 mmol) was added slowly and the reaction was allowed to stir at −78° C. for 1 hour. Trifluoroacetic anhydride (156 μL, 1.12 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.36, 5% EtOAc in Hexanes, [UV, KMnO₄]) to afford the product as an orange-yellow oil (67 mg, 0.42 mmol, 44%).

¹H NMR (500 MHz, CDCl₃) δ 7.42 (dd, J=15.0, 11.4 Hz, 1H), 6.65 (dd, J=14.8, 10.8 Hz, 1H), 6.29 (dd, J=14.8, 11.4 Hz, 1H), 6.16 (ddd, J=15.4, 10.8, 1.1 Hz, 1H), 5.98 (d, J=15.0 Hz, 1H), 5.92 (dd, J=15.2, 7.9 Hz, 2H), 5.89 (s, 1H), 2.18 (hept, J=6.8 Hz, 1H), 1.38 (p, J=7.3 Hz, 2H), 1.03 (d, J=6.8 Hz, 3H), 0.88 (t, J=7.4 Hz, 3H). ¹³C NMR (126 MHz, CDCl₃) δ 181.4, 179.8, 148.4, 144.5, 144.0, 128.4, 128.2, 123.2, 115.7, 95.1, 39.0, 29.5, 19.6, 11.7. ¹⁹F NMR (470 MHz, CDCl₃) δ −77.0.Mass spec: expected neutral mass for C₁₄H₁₇F₃O₂ (Da): 274.1181, observed neutral mass (Da): 274.1185, mass error (ppm) 1.7. UPLC Trace: Obtained using mobile phases of H₂O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then the 95% B from 7 to 7.1 minutes was on a gradient to 5% B until the end at 9 minutes. Note that NC-4 has multiple peaks in the UPLC trace due to the presence of a diketone tautomer and the hydrate. The purity was determined to be 95%.

### Example 14

### (S,4Z,6E,8E,10E)-1,1,1,2,2-pentafluoro-5-hydroxy-12-methyltetradeca-4,6,8,10-tetraen-3-one

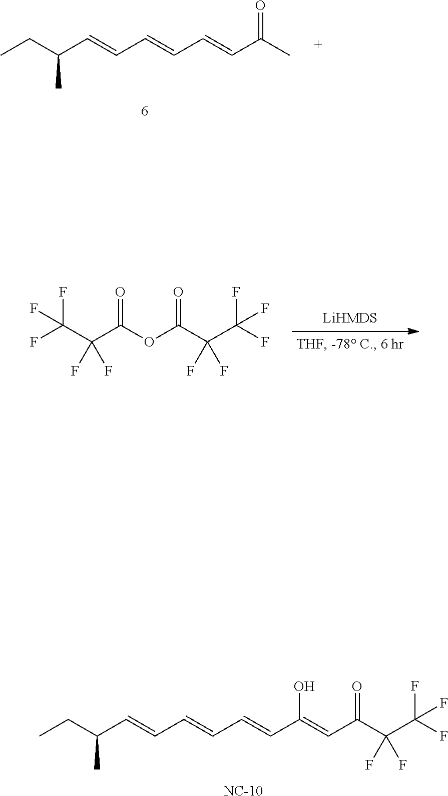

Ketone 6 (0.10 g, 0.56 mmol) was dissolved in anhydrous THF (7 mL) and cooled to −78° C. LiHMDS (1M in THF, 0.55 mL, 0.55 mmol) was added slowly and the reaction was allowed to stir at −78° C. for 1 hour. Pentafluoroethyl propionic anhydride (221 μL, 1.12 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.29, 2% EtOAc in Hexanes, [UV, KMnO₄]) to afford the product as an orange-yellow oil. ¹H NMR (500 MHz, CDCl₃) δ 7.44 (dd, J=15.0, 11.4 Hz, 1H), 6.66 (dd, J=14.8, 10.8 Hz, 1H), 6.29 (dd, J=14.8, 11.4 Hz, 1H), 6.17 (dd, J=15.2, 10.8 Hz, 1H), 5.98 (d, J=15.0 Hz, 1H), 5.96-5.90 (m, 2H), 2.18 (hept, J=7.0 Hz, 1H), 1.38 (p, J=7.4 Hz, 2H), 1.03 (d, J=6.7 Hz, 3H), 0.88 (t, J=7.4 Hz, 3H). ¹³C NMR (126 MHz, CDCl₃) δ 181.4, 181.2, 148.5, 144.8, 144.1, 128.4, 128.2, 123.1, 117.1, 107.7, 96.4, 39.0, 29.5, 19.6, 11.7. ¹⁹F NMR (470 MHz, CDCl₃) δ −82.6, −124.0.

Mass spec: expected neutral mass for C₁₅H17F502 (Da): 324.1148, observed neutral mass (Da): 324.1138, mass error (ppm) 5.0. UPLC Trace: Obtained using mobile phases of H₂O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then the 95% B from 7 to 7.1 minutes was on a gradient to 5% B until the end at 9 minutes. The purity was determined to be 95%.

### Example 14

### (S,3Z,5E,7E,9E)-1-chloro-1,1-difluoro-4-hydroxy-11-methyltrideca-3,5,7,9-tetraen-2-one

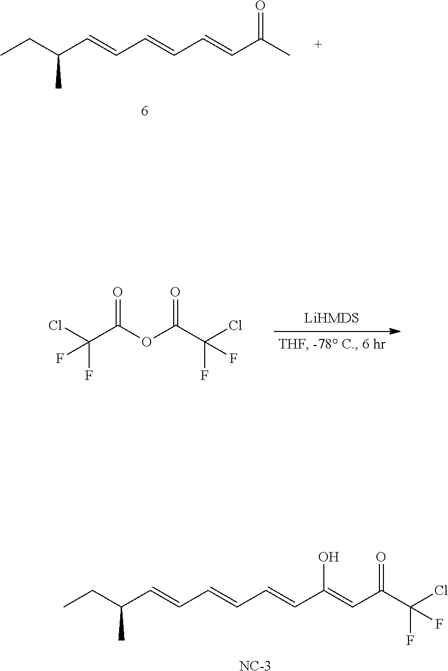

Ketone 6 (55 mg, 0.31 mmol) was dissolved in anhydrous THF (6 mL) and cooled to −78° C.

LiHMDS (1M in THF, 0.37 mL, 0.37 mmol) was added slowly and the reaction was allowed to stir at −78° C. for 1 hour. Chlorodifluoroacetic anhydride (221 μL, 1.12 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.40, 5% EtOAc in Hexanes, [UV, KMnO₄]) to afford the product as an orange-yellow oil. (54 mg, 0.19 mmol, 61%).

¹H NMR (500 MHz, CDCl₃) δ 7.40 (dd, J=15.0, 11.4 Hz, 1H), 6.64 (dd, J=14.8, 10.8 Hz, 1H), 6.28 (dd, J=14.8, 11.4 Hz, 1H), 6.16 (dd, J=15.2, 10.7 Hz, 1H), 5.98 (d, J=14.9 Hz, 1H), 5.92 (dd, J=15.2, 7.9 Hz, 1H), 5.86 (s, 1H), 2.17 (hept, J=7.0 Hz, 1H), 1.37 (p, J=7.3 Hz, 2H), 1.03 (d, J=6.7 Hz, 3H), 0.88 (t, J=7.4 Hz, 3H). ¹³C NMR (101 MHz, CDCl₃) δ 182.9, 180.1, 148.1, 143.9, 143.6, 128.3, 128.2, 123.0, 93.6, 38.8, 29.3, 19.5, 11.6. ¹⁹F NMR (470 MHz, CDCl₃) δ −65.7. Mass spec: expected neutral mass for C₁₄H₁₇ClF₂O₂ (Da): 290.08851, observed neutral mass (Da): 290.0892, mass error (ppm) 2.4. UPLC Trace: Obtained using mobile phases of H₂O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then, the 95% B from 7 to 7.1 minutes was on a gradient to 5% B until the end at 9 minutes. The purity was determined to be 95%.

### Example 15

### (S,3Z,5E,7E,9E)-4-hydroxy-11-methyltrideca-3,5,7,9-tetraen-2-one

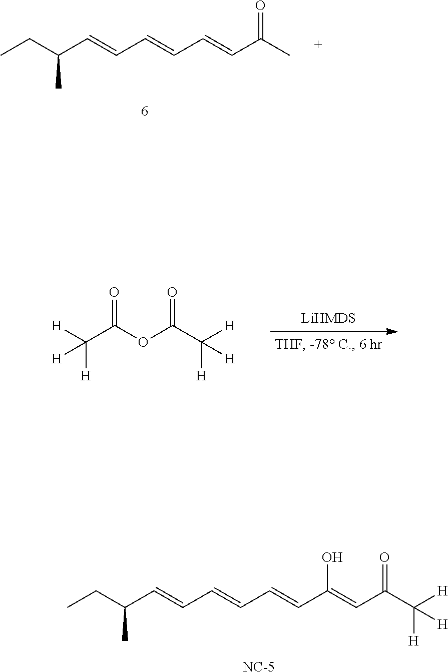

Ketone 6 (100 mg, 0.56 mmol) was dissolved in anhydrous THF (7 mL) and cooled to −78° C. LiHMDS (1M in THF, 0.62 mL, 0.62 mmol) was added slowly and the reaction was allowed to stir at −78° C. for 1 hour. Acetic anhydride (106 μL, 1.12 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.46, 7% EtOAc in Hexanes, [UV, KMnO₄]) to afford the product as an orange-yellow oil. (20 mg, 0.091 mmol, 16%). ¹H NMR (500 MHz, CDCl₃) δ 6.32-6.21 (m, 2H), 6.14 (dd, J=14.8, 11.0 Hz, 1H), 6.10-6.01 (m, 2H), 5.65 (dd, J=15.2, 7.8 Hz, 1H), 4.98 (s, 1H), 4.86 (s, 1H), 2.24 (s, 3H), 2.12 (hept, J=6.9 Hz, 1H), 1.33 (p, J=7.3 Hz, 2H), 1.00 (d, J=6.7 Hz, 3H), 0.85 (t, J=7.4 Hz, 3H). ¹³C NMR (126 MHz, CDCl₃) δ 168.7, 152.0, 142.9, 136.1, 130.6, 129.2, 128.6, 124.8, 105.0, 38.6, 29.7, 20.9, 19.9, 11.7. Mass spec: expected neutral mass for C₁₄H₂₀O₂ (Da): 220.1464, observed neutral mass (Da): 220.14633, mass error (ppm) 0.4.

### Example 16

### (S,3Z,5E,7E)-1,1,1-trichloro-4-hydroxy-9-methylundeca-3,5,7-trien-2-one

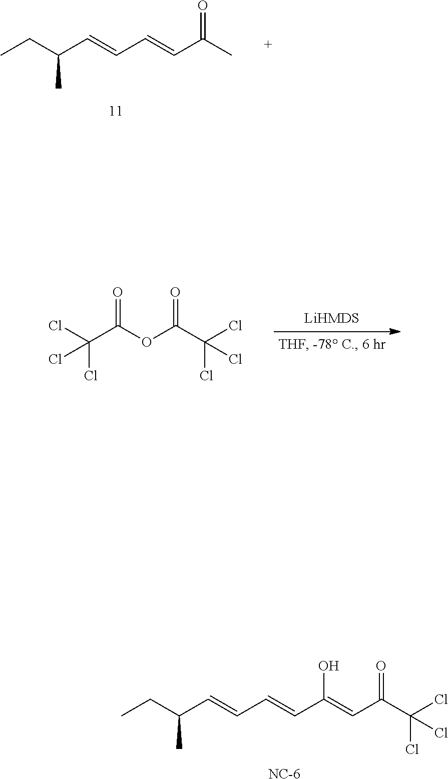 [SMILES: CC[C@H](C)/C=C/C=C/C(C)=O ; "11" ; O=C(OC(=O)C(Cl)(Cl)Cl)C(Cl)(Cl)Cl ; CC[C@H](C)/C=C/C=C/C(O)=C/C(=O)C(Cl)(Cl)Cl ; "NC-6"]

Ketone 11 (50 mg, 0.35 mmol) was dissolved in anhydrous THF (6 mL) and cooled to −78° C. LiHMDS (1M in THF, 0.42 mL, 0.42 mmol) was added slowly and the reaction was allowed to stir at −78° C. for 1 hour. Trichloroacetic anhydride (130 μL, 0.71 mmol) was then added slowly and the reaction was stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.45, 5% EtOAc in Hexanes, [UV, KMnO₄]) to afford the product as an orange-yellow oil. (58 mg, 0.20 mmol, 55%).

¹H NMR (400 MHz, CDCl₃) δ 7.32 (dd, J=15.2, 10.4 Hz, 1H), 6.24-6.06 (m, 3H), 5.96 (d, J=15.2 Hz, 1H), 2.21 (hept, J=6.8 Hz, 1H), 1.39 (p, J=7.4 Hz, 2H), 1.05 (d, J=6.7 Hz, 3H), 0.88 (t, J=7.4 Hz, 3H). ¹³C NMR (101 MHz, CDCl₃) δ 186.0, 178.3, 152.0, 143.0, 127.2, 122.5, 92.8, 39.0, 29.2, 19.4, 11.6. Mass spec: expected neutral mass for C₁₂H₁₅Cl₃O₂ (Da): 296.01376, observed neutral mass (Da): 296.0158, mass error (ppm) 6.2. UPLC Trace: Obtained using mobile phases of H₂O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then the 95% B from 7 to 7.1 minutes was on a gradient to 5% B until the end at 9 minutes. The purity was determined to be 96%.

### Example 17

### (S,3Z,5E,7E)-1,1,1-trifluoro-4-hydroxy-9-methylundeca-3,5,7-trien-2-one

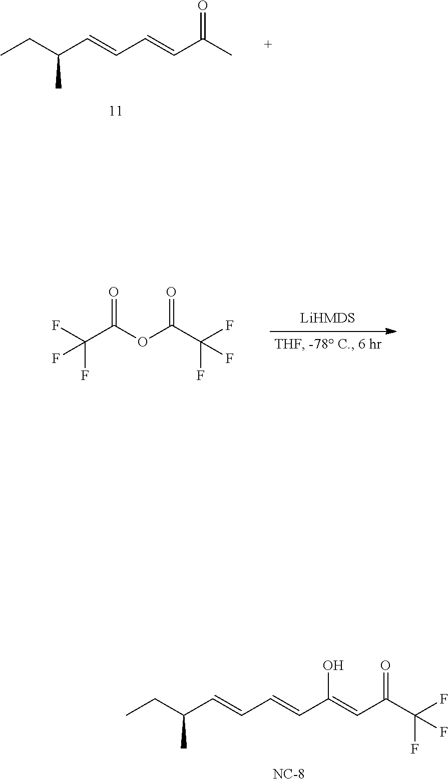 [SMILES: CC[C@H](C)/C=C/C=C/C(C)=O ; "11" ; O=C(OC(=O)C(F)(F)F)C(F)(F)F ; CC[C@H](C)/C=C/C=C/C(O)=C/C(=O)C(F)(F)F ; "NC-8"]

Ketone 11 (100 mg, 0.66 mmol) was dissolved in anhydrous THF (8 mL) and cooled to −78° C. LiHMDS (1M in THF, 0.62 mL, 0.62 mmol) was added slowly and the reaction was allowed to stir at −78° C. for 1 hour. Trifluoroacetic anhydride (365 μL, 2.63 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography twice (Rf=0.26, 2% EtOAc in Hexanes, [UV, KMnO₄]) to afford the product as an orange-yellow oil. (21 mg, 0.085 mmol, 13%). ¹H NMR (400 MHz, CDCl₃) δ 7.37 (dd, J=15.2, 10.0 Hz, 1H), 6.26-6.11 (m, 2H), 5.94 (d, J=15.2 Hz, 1H), 5.89 (s, 1H), 2.22 (hept, J=6.8 Hz, 1H), 1.40 (p, J=7.5 Hz, 2H), 1.05 (d, J=6.7 Hz, 3H), 0.88 (t, J=7.4 Hz, 3H).¹³C NMR (126 MHz, CDCl₃) δ 181.9, 180.0, 153.3, 144.9, 127.3, 122.6, 115.7, 95.0, 39.1, 29.3, 19.4, 11.7.¹⁹F NMR (470 MHz, CDCl₃) δ−77.0. Mass spec: expected neutral mass for C₁₂H15F302 (Da): 248.10241, observed neutral mass (Da): 248.1035, mass error (ppm) 4.5.

### Example 18

### (S,3Z,5E,7E)-1-chloro-1,1-difluoro-4-hydroxy-9-methylundeca-3,5,7-trien-2-one

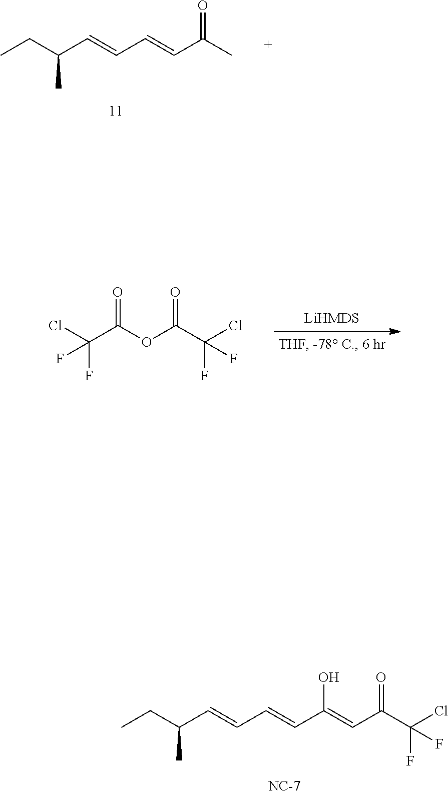 [SMILES: CC[C@H](C)/C=C/C=C/C(C)=O ; "11" ; O=C(OC(=O)C(F)(F)Cl)C(F)(F)Cl ; CC[C@H](C)/C=C/C=C/C(O)=C/C(=O)C(F)(F)Cl ; "NC-7"]

Ketone 11 (100 mg, 0.66 mmol) was dissolved in anhydrous THF (8 mL) and cooled to −78° C. LiHMDS (1M in THF, 0.66 mL, 0.66 mmol) was added slowly and the reaction was allowed to stir at −78° C. for 1 hour. Chlorodifluoroacetic anhydride (456 μL, 2.63 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography twice (Rf=0.29, 5% EtOAc in Hexanes, [UV, KMnO₄]) to afford the product as an orange-yellow oil. (26 mg, 0.098 mmol, 15%). ¹H NMR (500 MHz, CDCl₃) δ 7.36 (dd, J=15.2, 10.1 Hz, 1H), 6.25-6.11 (m, 2H), 5.94 (d, J=15.2 Hz, 1H), 5.86 (s, 1H), 2.22 (hept, J=6.8 Hz, 1H), 1.40 (p, J=7.5 Hz, 2H), 1.05 (d, J=6.7 Hz, 3H), 0.88 (t, J=7.4 Hz, 3H). ¹³C NMR (126 MHz, CDCl₃) δ 183.05, 180.75, 152.97, 144.38, 127.31, 122.48, 120.26 (t, J=300.6 Hz), 93.51, 39.13, 29.30, 19.42, 11.70. ¹⁹F NMR (470 MHz, CDCl₃) δ−65.7. Mass spec: expected neutral mass for C₁₂H₁₅ClF₂O₂ (Da): 264.07286, observed neutral mass (Da): 264.0724, mass error (ppm) −1.7.

### Example 19

### (S,3Z,5E,7E,9E)-1-chloro-1,1-difluoro-4-hydroxy-11-methyltrideca-3,5,7,9-tetraen-2-one

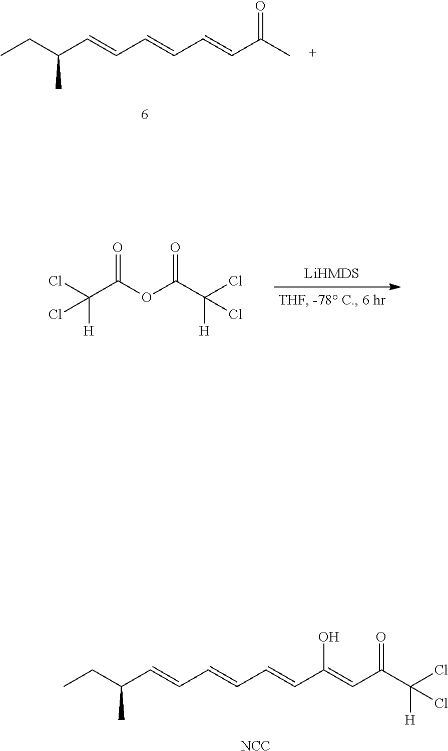

Ketone 6 (0.10 g, 0.56 mmol) was dissolved in anhydrous THF (7 mL) and cooled to −78° C. LiHMDS (1M in THF, 0.62 mL, 0.62 mmol) was added slowly and the reaction was allowed to stir at −78° C. for 1 hour. Dichloroacetyl chloride (108 μL, 1.12 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.53, 5% EtOAc in Hexanes, [UV, KMnO₄]) to afford the product as a yellow oil (97 mg, 0.34 mmol, 60%). ¹H NMR (400 MHz, CDCl₃) δ 7.33 (ddd, J=15.1, 11.3, 0.8 Hz, 1H), 6.59 (dd, J=14.8, 10.8 Hz, 1H), 6.27 (dd, J=14.8, 11.3 Hz, 1H), 6.14 (dd, J=15.2, 10.8 Hz, 1H), 6.03-5.93 (m, 2H), 5.93-5.79 (m, 2H), 2.16 (hept, J=6.9 Hz, 1H), 1.37 (p, J=7.2 Hz, 2H), 1.02 (d, J=6.7 Hz, 3H), 0.87 (t, J=7.4 Hz, 3H). ¹³C NMR (101 MHz, CDCl₃) δ 189.6, 178.3, 147.3, 142.5, 142.4, 128.3, 128.3, 123.4, 95.2, 68.8, 38.8, 29.4, 19.6, 11.6. Mass spec: expected neutral mass for C₁₄H₁₈C₂O₂ (Da): 288.06839, observed neutral mass (Da): 288.0697, mass error (ppm) 4.4. UPLC Trace: Obtained using mobile phases of H₂O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then the 95% B from 7 to 7.1 minutes was on a gradient to 5% B until the end at 9 minutes. The purity was determined to be 99%.

### Example 20

### (S,3Z,5E,7E,9E,11E)-1,1-1-trichloro-4-hydroxy-13-methyltpentadeca-3,5,7,9,11-pentaene-2-one

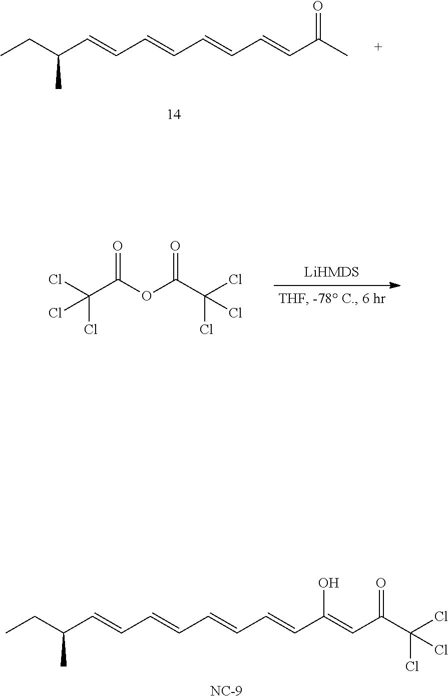

Ketone 14 (100 mg, 0.50 mmol) was dissolved in anhydrous THF (8 mL) and cooled to −78° C. LiHMDS (1M in THF, 0.60 mL, 0.60 mmol) was added slowly and the reaction was allowed to stir at −78° C. for 1 hour. Trichloroacetic anhydride (183 μL, 1 mmol) was then added slowly and the reaction stirred for 5 hours at −78° C. The reaction was diluted with diethyl ether (40 mL), allowed to warm to 0° C., and was quenched by the addition of sat. aq. NH₄Cl (50 mL), and allowed to warm to rt. The organics were washed with sat. aq. NaHCO₃ (3×20 mL), sat. aq. NH₄Cl (3×20 mL), and brine (2×20 mL). The organics were dried with Na₂SO₄, filtered, and concentrated. The crude product was purified via flash chromatography (Rf=0.64, 5% EtOAc in Hexanes, [UV, KMnO₄]) to afford the product as an orange-yellow oil. (134 mg, 0.38 mmol, 77%). ¹H NMR (400 MHz, CDCl₃) δ 7.38 (dd, J=15.0, 11.5 Hz, 1H), 6.66 (dd, J=14.7, 11.1 Hz, 1H), 6.44 (dd, J=14.8, 10.6 Hz, 1H), 6.33 (dd, J=14.7, 11.4 Hz, 1H), 6.25 (dd, J=14.8, 11.0 Hz, 1H), 6.20-6.06 (m, 2H), 6.01 (d, J=15.0 Hz, 1H), 5.79 (dd, J=15.2, 7.9 Hz, 1H), 2.14 (hept, J=7.0 Hz, 1H), 1.35 (p, J=7.2 Hz, 2H), 1.02 (d, J=6.8 Hz, 3H), 0.87 (t, J=7.4 Hz, 3H). ¹³C NMR (101 MHz, CDCl₃) δ 185.9, 177.8, 145.3, 142.6, 142.5, 139.0, 129.7, 129.4, 128.5, 123.3, 95.1, 93.1, 38.7, 29.5, 19.7, 11.6. Mass spec: expected neutral mass for C₁₆H₁₉Cl₃O₂ (Da): 348.04506, observed neutral mass (Da): 348.049107, mass error (ppm) 3.7. UPLC Trace: Obtained using mobile phases of H2O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then the 95% B from 7 to 7.1 minutes was on a gradient to 5% B until the end at 9 minutes. The purity was determined to be 95%.

### Example 21

### (S,3Z,5E,7E,9E)-1,1-dichloro-1-fluoro-4-hydroxy-11-methyltrideca-3,5,7,9-tetraene-2-one (NC-2)

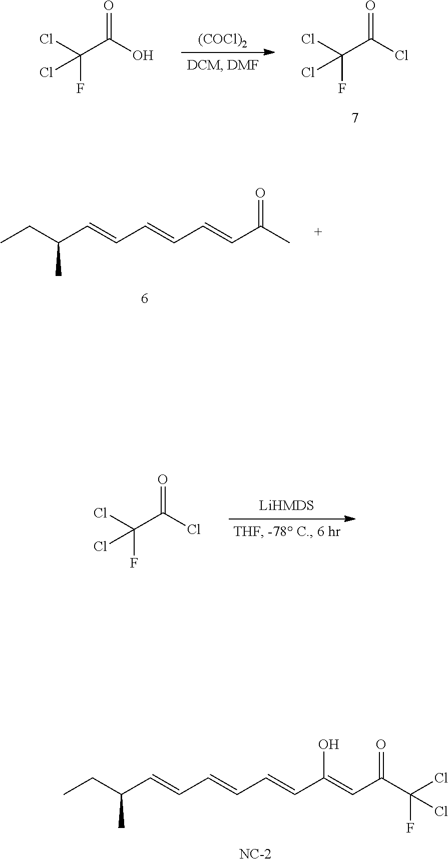

Ketone 6 (0.1 g, 0.56 mmol) was dissolved in anhydrous THF (7 mL) in a flame-dried flask and cooled to −78° C. LiHMDS (1M in THF, 0.62 mL, 0.62 mmol) was added slowly, and the reaction was stirred for 1 hour at −78° C. In parallel, dichlorofluoro acetic acid (0.19 mL, 2.24 mmol) was dissolved in dry DCM (5 mL) and cooled to 0° C. DMF (6 μL) was added followed by dropwise addition of oxalyl chloride (0.192 mL, 2.24 mmol) at 0° C. under an inert nitrogen atmosphere. The reaction was allowed to warm to room temperature and stirred for 1 hour to form the acid chloride, upon which the DCM was removed under a stream of nitrogen. Then acid chloride 7 was added to the formed enolate at −78° C. dropwise, and the reaction was stirred for 3 hours. Upon completion, the reaction was diluted with diethyl ether (40 mL), allowed to warm to 0° C., and was quenched by the addition of sat. aq. NH₄Cl (50 mL), and allowed to warm to rt. The organics were washed with sat. aq. NH₄Cl (3×20 mL), sat. aq. NaHCO₃ (3×20 mL), and brine (2×20 mL). The organics were dried with Na₂SO₄, filtered, and concentrated. The crude product was purified via flash chromatography (95:5 Hex/EtOAc) to afford the product as an orange oil. ¹H NMR (500 MHz, CDCl₃) δ 7.39 (dd, J=15.0, 11.3 Hz, 1H), 6.63 (dd, J=14.8, 10.7 Hz, 1H), 6.28 (dd, J=14.8, 11.4 Hz, 1H), 6.16 (dd, J=15.2, 10.8 Hz, 1H), 5.98 (d, J=15.0 Hz, 1H), 5.95-5.82 (m, 2H), 2.17 (hept, J=6.9 Hz, 1H), 1.38 (p, J=7.4 Hz, 2H), 1.03 (d, J=6.7 Hz, 3H), 0.88 (t, J=7.4 Hz, 3H). ¹³C NMR (126 MHz, CDCl₃) δ 185.6, 185.4, 179.3, 148.0, 143.5, 143.4, 128.4, 128.3, 123.2, 115.2, 92.8, 38.9, 29.5, 19.7, 11.7. ¹⁹F NMR (470 MHz, CDCl₃) δ−67.5.

Mass spec: expected neutral mass for C₁₄H₁₇Cl₂FO₂ (Da): 306.05896, observed neutral mass (Da): 306.0606, mass error (ppm) 5.4. UPLC Trace: Obtained using mobile phases of H₂O+0.1% formic acid (A) and acetonitrile+0.1% formic acid (B). Samples were eluted using a gradient mode with mobile phase B ranging from 5% to 95% over 6 minutes at a flow rate of 0.5 mL/min. Then, the 95% B from 7 to 7.1 minutes was on a gradient to 5% B until the end at 9 minutes. The purity was determined to be 95%.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

What is claimed is:

1. A compound selected from: 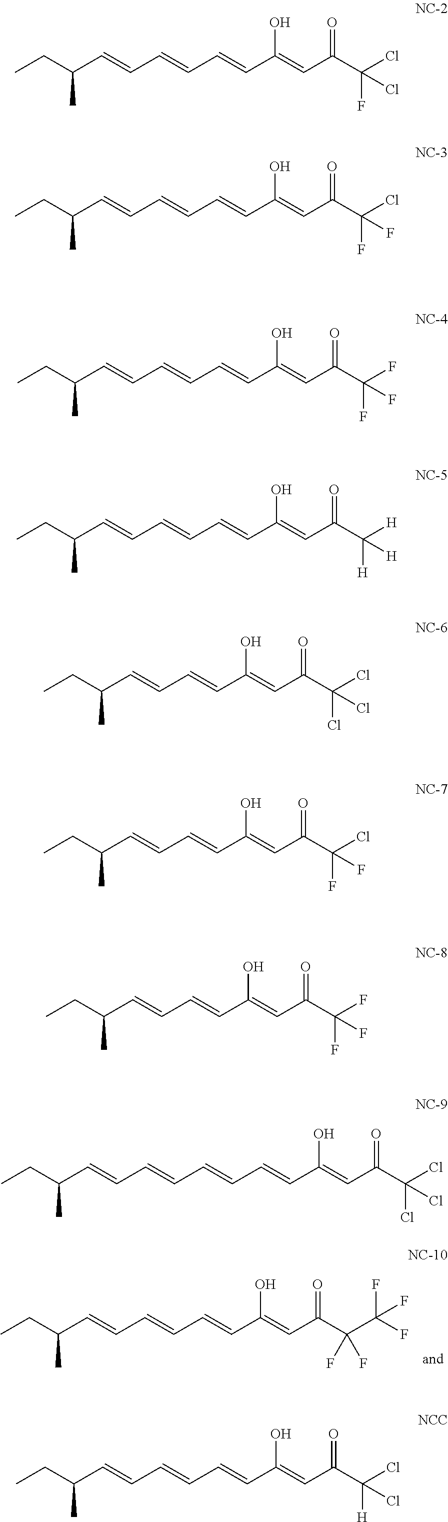
or a pharmaceutically acceptable salt or hydrate thereof.

2. A pharmaceutical composition comprising a compound selected from: 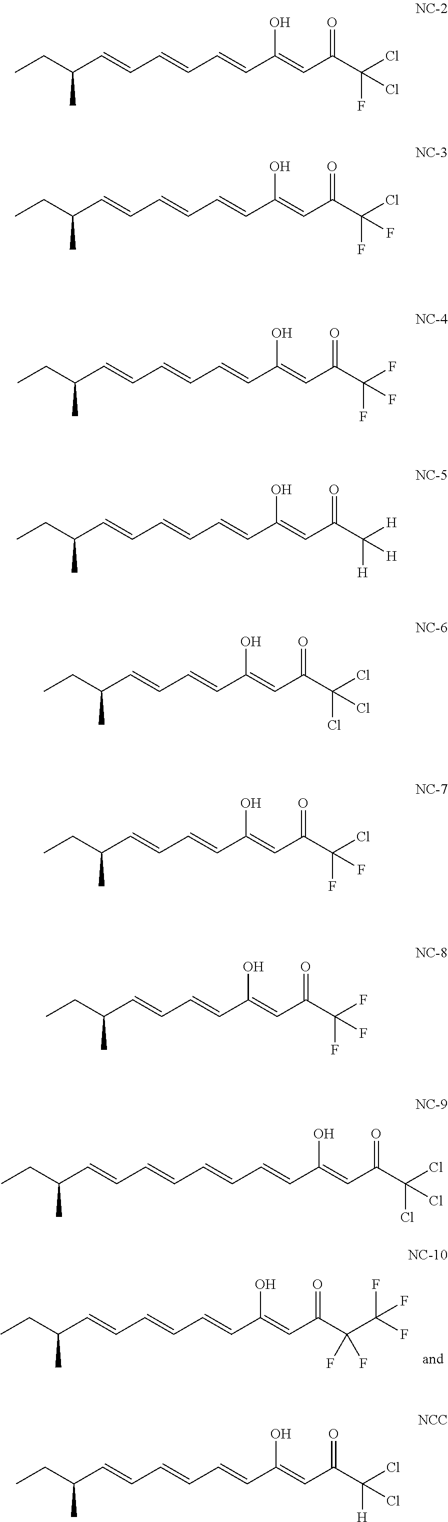
or a pharmaceutically acceptable salt or hydrate thereof and a pharmaceutically acceptable carrier, excipient, or diluent.

3. The pharmaceutical composition of claim 2, which further comprises another compound, which is a therapeutic agent.

4. A method of inhibiting vesicle amine transport-1 (VAT1) in a patient in need thereof, which method comprises administering to the patient an inhibitory effective amount of a pharmaceutical composition of claim 2, whereupon the VAT1 is inhibited in the patient.

5. The method of claim 4, wherein the patient has cancer.

6. The method of claim 5, wherein the cancer is breast cancer, glioblastoma, hepatocellular carcinoma, or angiosarcoma.

7. The method of claim 4, wherein the pharmaceutical composition is administered orally or parenterally.

8. The method of claim 5, wherein metastasis of the cancer is inhibited.

9. A compound of the formula: 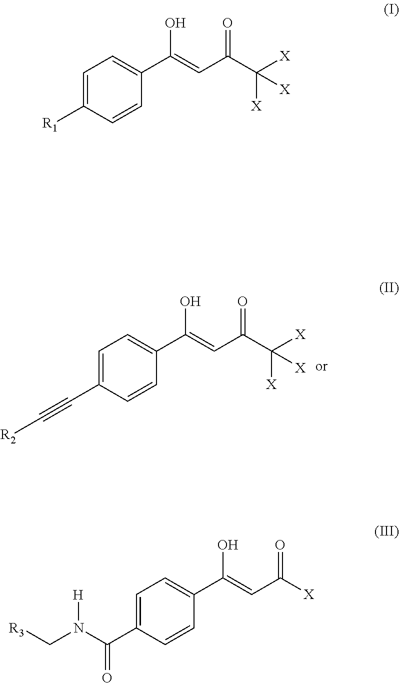
or a pharmaceutically acceptable salt or hydrate thereof
wherein:
each R₁, R₂, and R₃ is independently C₁-C₆ alkyl, aryl, heteroaryl, or heterocyclyl, wherein each R₁, R₂, and R₃ can be optionally independently substituted with at least one of alkyl, aryl, heteroaryl, and heterocyclyl; and
X is hydrogen or halogen.

10. The compound of claim 9, wherein the R₁ is:

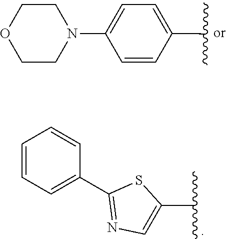 [SMILES: *c1ccc(N2CCOCC2)cc1 ; *c1cnc(-c2ccccc2)s1]

11. The compound of claim 9, wherein the R₂ is:

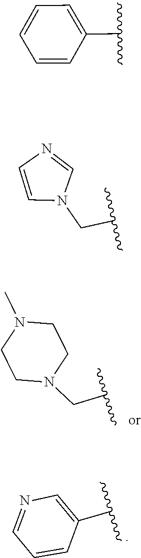

12. The compound of claim 9, wherein the R₃ is:

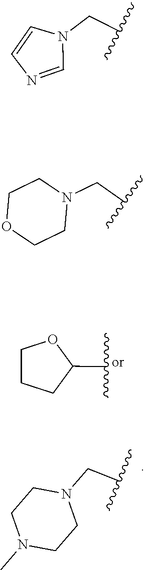

13. The compound of claim 9, wherein X is halogen.
